(19) United States
(12) Patent Application Publication
(10) Pub. No.: US 2016/0030806 A1
(43) Pub. Date: Feb. 4, 2016

(54) EXERCISE ABILITY EVALUATION METHOD, EXERCISE ABILITY EVALUATION APPARATUS, EXERCISE ABILITY CALCULATION METHOD, AND EXERCISE ABILITY CALCULATION APPARATUS

(71) Applicant: Seiko Epson Corporation, Tokyo (JP)

(72) Inventor: Kazumi MATSUMOTO, Shiojiri-shi (JP)

(21) Appl. No.: 14/811,627

(22) Filed: Jul. 28, 2015

(30) Foreign Application Priority Data
Jul. 31, 2014 (JP) 2014-155939
Jul. 31, 2014 (JP) 2014-156772

(51) Int. Cl. A63B 24/00; A61B 5/22; A61B 5/029; A61B 5/00; A61B 5/0205

(52) U.S. Cl. CPC A63B 24/0062; A61B 5/4519; A61B 5/0205; A61B 5/029; A61B 5/7246; A61B 5/7278; A61B 5/222; A63B 2024/0068; A63B 2024/0065; A61B 2562/0219

(57) ABSTRACT

An exercise ability evaluation method includes: acquiring measurement data of whole body endurance of a user at a time of an exercise; acquiring reference data of the whole body endurance; comparing the measurement data to the reference data; and evaluating a muscle ability of the user based on a comparison result obtained by comparing the measurement data and the reference data and generating exercise ability evaluation information.

### BACKGROUND

1. Technical Field

The present invention relates to an exercise ability evaluation method, an exercise ability evaluation apparatus, an exercise ability calculation method, and an exercise ability calculation apparatus.

2. Related Art

In the related art, methods of estimating and showing physical strengths or exercise abilities of users based on results obtained by measuring biological information at the time of exercise of the user have been introduced. For example, JP-A-2011-161079 introduces a method of calculating the maximum value of a pulse measured by a pulsimeter within a predetermined measurement time and a relative heart rate reserve based on a pulse rate at rest after a user starts to walk, correcting a moved walking distance at a measurement time by the relative heart rate reserve, and calculating a physical strength age by coefficients set in correspondence to the corrected walking distance and sex.

JP-A-2004-113821 discloses a method of calculating a running distance of the subject per unit time by multiplying a stride of a subject (user) and a pitch detected in an immediately previous step, obtaining an exercise intensity [W] by multiplying the running distance by the weight of the subject stored in a RAM, converting the exercise intensity into [kpm/minute], and calculating an exercise intensity per unit time.

As elements improving an exercise ability (sports ability), there are three elements, that is, a cardiorespiratory ability, a muscle ability, and a technical skill. In recent years, various methods of evaluating the cardiorespiratory ability and the technical skill through exercise analysis technologies have been introduced. However, in particular, there are no methods of simply obtaining the muscle ability interesting athletes or sports enthusiasts. In an exercise ability evaluation method (exercise index measurement method) disclosed in JP-A-2011-161079, content about calculation of physical strength ages based on a heart rate and evaluation of muscle abilities have not been described either.

For example, when an exercise amount of a running, a walking race, or the like is measured, an exercise intensity indicating the intensity of an exercise at a certain instant (unit time) and an exercise output indicating the amount of an exercise within a certain time can be considered. When an exercise ability of a subject is calculated, it is effective to calculate an exercise ability based on the exercise output. In JP-A-2004-113821, however, a method of calculating an exercise intensity has been described, but a method of obtaining the exercise output based on sensing data by an inertial sensor has not been disclosed.

### SUMMARY

An advantage of some aspects of the invention is to solve at least a part of the problems described above, and the invention can be implemented as the following forms and application examples.

### Application Example 1

An exercise ability evaluation method according to this application example includes: acquiring measurement data of whole body endurance of a user; acquiring reference data of the whole body endurance; comparing the measurement data to the reference data; and evaluating a muscle ability of the user based on a comparison result obtained by comparing the measurement data and the reference data and generating exercise ability evaluation information.

According to the exercise ability evaluation method of this application example, the superiority or inferiority of the muscle ability of the user or the degree of the muscle ability of the user can be evaluated and generated as the exercise ability evaluation information based on the comparison result between the reference data of the whole body endurance and the measurement data of the whole body endurance of the user. Thus, it is possible to obtain the evaluation of the muscle ability relatively easily using the data of the whole body endurance.

### Application Example 2

This application example is directed to the exercise ability evaluation method according to the application example described above, wherein an index of the whole body endurance includes at least one of a cardiorespiratory ability, a lactic acid value, a degree of fatigue and an oxygen intake amount of the user.

According to this application example, it is possible to generate the exercise ability evaluation information using at least one of a cardiorespiratory ability, a lactic acid value, and a degree of fatigue, and an oxygen intake amount of the user as the whole body endurance. Thus, it is possible to obtain the evaluation of the muscle ability using the index of the previous whole body endurance or a method of calculating the index.

### Application Example 3

This application example is directed to the exercise ability evaluation method according to the application example described above, wherein the index is a running time per predetermined distance of the user.

According to this application example, it is possible to generate the exercise ability evaluation information using the running time of the user per predetermined distance as the index of the whole body endurance. Thus, for example, by calculating the oxygen intake amount from the running time per predetermined distance, it is possible to obtain the evaluation of the muscle ability relatively easily.

### Application Example 4

This application example is directed to the exercise ability evaluation method according to the application example described above, wherein the index is the oxygen intake amount, and the oxygen intake amount is measured through a running test within a predetermined time or a running test of reciprocation of a predetermined distance.

According to this application example, the oxygen intake amount is measured by a running test within a predetermined time or a running test of reciprocation of a predetermined distance. Thus, for example, it is possible to obtain the evaluation of the muscle ability from the oxygen intake amount measured in a general running test such as 12-minute running, 15-minute running, or a shuttle run of reciprocation of a predetermined distance.

### Application Example 5

This application example is directed to the exercise ability evaluation method according to the application example described above, wherein the index is the oxygen intake amount, and the oxygen intake amount is calculated based on pulse data and body movement data of the user.

According to this application example, since the oxygen intake amount can be calculated by pulse rate data and body movement data of the user, it is possible to obtain the exercise ability evaluation information (exercise ability evaluation data) with higher accuracy.

### Application Example 6

This application example is directed to the exercise ability evaluation method according to the application example described above, wherein the oxygen intake amount serving as the reference data is acquired based on a heart rate at a time of rest and a maximum heart rate (HRₘₐₓ).

According to this application example, since the oxygen intake amount serving as the reference data can be acquired from the heart rate at the time of rest and the maximum heart rate, it is possible to obtain the oxygen intake amount relatively easily without causing the user to do unreasonable work (exercise).

### Application Example 7

This application example is directed to the exercise ability evaluation method according to the application example described above, wherein the muscle ability of the user is evaluated based on a difference between the measurement data and the reference data and the exercise ability evaluation information is generated.

According to this application example, it is possible to generate the exercise ability evaluation information of the user relatively easily.

### Application Example 8

An exercise ability evaluation apparatus according to this application example includes: a storage unit that stores reference data of whole body endurance; and an exercise ability evaluation information generation unit that generates exercise ability evaluation information by evaluating a muscle ability of a user based on a comparison result obtained by comparing measurement data of whole body endurance of the user and the reference data.

According to the exercise ability evaluation apparatus of this application example, the exercise ability evaluation information generation unit evaluates the superiority or inferiority of the muscle ability of the user or the degree of the muscle ability of the user based on the comparison result between the reference data of the whole body endurance stored in the storage unit and the measurement data of the whole body endurance of the user, and generates the exercise ability evaluation information. Thus, it is possible to obtain the evaluation of the muscle ability relatively easily using the data of the whole body endurance.

### Application Example 9

An exercise ability evaluation system according to this application example includes a measurement device measuring measurement data of a whole body endurance of a user and an exercise ability evaluation apparatus including a measurement data acquisition unit that acquires the measurement data measured by the measurement device, a storage unit that stores reference data of the whole body endurance, and an exercise ability evaluation information generation unit that generates exercise ability evaluation information of the user based on a comparison result between the measurement data and the reference data.

According to this application example, it is possible to provide the exercise ability evaluation system in which the exercise ability evaluation apparatus can evaluate the superiority or inferiority of the muscle ability of the user or the degree of the muscle ability of the user with respect to the reference data based on the comparison result between the reference data of the whole body endurance stored in the storage unit and the measurement data of the whole body endurance of the user measured by the measurement device such as a sensor or a pulsimeter, and can generate the exercise ability evaluation information.

### Application Example 10

A program according to this application example causes a computer to obtain a comparison result obtained by comparing the acquired measurement data of the whole body endurance of the user and the reference data of the whole body endurance, evaluate the muscle ability of the user based on the comparison result, and generate the exercise ability evaluation information.

According to this application example, it is possible to the program that evaluate the superiority or inferiority of the muscle ability of the user or the degree of the muscle ability of the user based on the comparison result between the reference data of the whole body endurance and the measurement data of the whole body endurance of the user, and generate the exercise ability evaluation information.

### Application Example 11

An exercise ability calculation method according to this application example includes acquiring information regarding the acceleration and the velocity from an output of an inertial sensor worn by a subject, acquiring information regarding a weight of the subject, and calculating an exercise output indicating an exercise amount within a predetermined time from the information regarding the acceleration, the velocity, and the weight.

According to the exercise ability calculation method of this application example, it is possible to obtain the exercise output indicating the exercise amount of the subject within a predetermined time with high accuracy from the information regarding the acceleration and the velocity acquired by the inertial sensor worn by the subject and the information regarding the weight of the subject, and to calculate the exercise ability of the subject based on the obtained exercise output.

### Application Example 12

This application example is directed to the exercise ability calculation method according to the application example described above, which further includes acquiring information regarding a pulse rate of the subject and calculating a cardiac output based on the pulse rate and the exercise output.

According to this application example, it is possible to calculate the cardiac output based on the acquired pulse rate of the subject and the exercise output. Therefore, it is possible to obtain the exercise ability of the subject using the cardiac output.

In the following application examples including this application example, the “pulse rate” is assumed to include a “heart rate.”

### Application Example 13

This application example is directed to the exercise ability calculation method according to the application example described above, wherein information regarding the pulse rate is a pulse rate measured by a pulsimeter worn by the subject.

According to this application example, since information regarding the pulse rate of the subject and the information regarding the acceleration and the velocity can be acquired in real time, the exercise ability of the subject can be obtained with high accuracy.

### Application Example 14

This application example is directed to the exercise ability calculation method according to the application example described above, wherein the cardiac output is estimated and calculated based on a value obtained by dividing the exercise output by the pulse rate.

According to this application example, it is possible to relatively easily estimate and calculate the cardiac output, for which a special equipment or mechanism for measurement is normally necessary, from the exercise output calculated based on information or the like acquired by the inertial sensor and the measured pulse rate. Further, it is possible to calculate the exercise ability of the subject from the obtained cardiac output.

### Application Example 15

This application example is directed to the exercise ability calculation method according to the application example described above, wherein an exercise ability is calculated based on a comparison result obtained by comparing the calculated cardiac output to a reference cardiac output to be compared to the cardiac output.

According to this application example, for example, by comparing cardiac outputs of the subject and the same generation or a past cardiac output of the subject to a cardiac output calculated as a reference cardiac output, it is possible to determine whether the exercise ability is improved or degraded with respect to a reference value or calculate the degree of exercise ability or the like.

### Application Example 16

This application example is directed to the exercise ability calculation method according to the application example described above, wherein an exercise ability is calculated based on a result obtained by comparing first data indicating a relation between the pulse rate and the exercise output to second data indicating a relation between a different pulse rate from the first data and the exercise output.

According to this application example, the exercise ability of the subject can be evaluated relatively since the first data indicating the relation between the pulse rate of the subject and the exercise output is compared to, for example, the second data indicating the relation between the exercise output and the past pulse rate of the same generation as the user or the subject.

### Application Example 17

This application example is directed to the exercise ability calculation method according to the application example described above, wherein the exercise outputs of the first data and the second data are compared at a predetermined pulse rate.

According to this application example, the exercise ability can be determined to be higher as the exercise outputs in the same pulse rate are high. Therefore, by comparing the exercise outputs, it is possible to relatively comprehend the exercise ability (the first data) of the subject to the exercise ability of the second data.

### Application Example 18

This application example is directed to the exercise ability calculation method according to the application example described above, wherein pulse rates of the first data and the second data are compared for a predetermined exercise output.

According to this application example, the exercise ability can be determined to be higher as the pulse rates at the same exercise output are low. Therefore, the subject can relatively comprehend the exercise ability (the first data) of the subject with respect to the exercise ability of the second data by comparing the pulse rates.

### Application Example 19

This application example is directed to the exercise ability calculation method according to the application example described above, wherein pulse rates at which the exercise outputs of the first data and the second data are saturated are compared.

According to this application example, the exercise ability can be determined to be lower in that the exercise output is not set to appropriate for the pulse rate as the pulse rate at the time of the saturation of the exercise output is lower. Accordingly, the subject can relatively comprehend the exercise ability (the first data) of the subject with respect with respect to the exercise ability of the second data by comparing the pulse rates at which the exercise output is saturated.

### Application Example 20

This application example is directed to the exercise ability calculation method according to the application example described above, wherein maximum pulse rates of the first data and the second data are compared.

According to this application example, the exercise ability can be determined to be higher as the maximum pulse rate is higher. Therefore, the subject can relatively comprehend the exercise ability (the first data) of the subject with respect to the exercise ability of the second data by comparing the maximum pulse rates.

### Application Example 21

This application example is directed to the exercise ability calculation method according to the application example described above, wherein an inclination of a non-measurement section is estimated from an inclination of an actual measurement section of a graph indicating a relation between the pulse rate and the exercise output, and the exercise output at a predetermined pulse rate is estimated.

According to this application example, since the relation between the exercise output and the pulse rate of the non-measurement section can be estimated, the exercise output at the predetermined pulse rate can be estimated including the relation between the exercise output and the pulse rate of the non-measurement section. Thus, it is possible to calculate the exercise ability of the subject with high accuracy.

### Application Example 22

This application example is directed to the exercise ability calculation method according to Application Example 21, wherein a maximum oxygen intake amount is estimated and calculated based on the estimated exercise output.

According to this application example, it is possible to estimate and calculate the oxygen amount necessary for the maximum value of the estimated exercise output as the maximum oxygen intake amount.

### Application Example 23

This application example is directed to the exercise ability calculation method according to Application Example 21, wherein the predetermined pulse rate is a maximum pulse rate of the subject.

According to this application example, since the predetermined pulse rate is the maximum pulse rate of the subject, it is possible to estimate the oxygen amount necessary for the maximum exercise output estimation value as the maximum oxygen intake amount, which is an effective index of the exercise ability evaluation, using the exercise output corresponding to the maximum pulse rate as the maximum exercise output estimation value.

### Application Example 24

An exercise ability calculation apparatus according to this application example includes a reception unit that acquires information regarding acceleration and velocity from an output of an inertial sensor worn by a subject and information regarding a weight of the subject, and a calculation unit that calculates an exercise output indicating an exercise amount within a predetermined time from the information regarding the acceleration, the velocity, and the weight.

According to this application example, it is possible to provide the exercise ability calculation apparatus capable of obtaining the exercise output indicating the exercise amount of the subject within the predetermined time with high accuracy from the information regarding the acceleration and the velocity acquired by the inertial sensor worn by the subject and the information regarding the weight of the subject, and calculating the exercise ability of the subject based on the obtained exercise output.

### Application Example 25

This application example is directed to the exercise ability calculation apparatus according to the application example described above, wherein the reception unit includes a function of acquiring information regarding a pulse rate of the subject, and the calculation unit includes a function of calculating a cardiac output based on the pulse rate and the exercise output.

According to this application example, it is possible to calculate the cardiac output based on the exercise ability and the pulse rate of the subject and obtain the exercise ability of the subject using the cardiac output.

### Application Example 26

An exercise ability calculation system according to this application example includes an inertial sensor worn by a subject, and an exercise ability calculation apparatus including: a reception unit that acquires information regarding acceleration and velocity from an output of the inertial sensor and information regarding a weight of the subject; and a calculation unit that calculates an exercise output indicating an exercise amount within a predetermined time from the information regarding the acceleration, the velocity, and the weight.

According to this application example, it is possible to provide the exercise ability calculation system capable of obtaining the exercise output indicating the exercise amount of the subject within the predetermined time with high accuracy from the information regarding the acceleration and the velocity acquired by the inertial sensor worn by the subject and the information regarding the weight of the subject, and calculating the exercise ability of the subject based on the obtained exercise output.

### Application Example 27

This application example is directed to the exercise ability calculation system according to the application example described above, wherein the exercise ability calculation system includes a pulsimeter that is worn by the subject and measures a pulse rate of the subject, and the calculation unit has a function of calculating a cardiac output based on the pulse rate and the exercise output.

According to this application example, it is possible to calculate the cardiac output based on the exercise ability and the pulse rate of the subject and obtain the exercise ability of the subject using the cardiac output.

### Application Example 28

A program according to this application example causes a computer to obtain information regarding acceleration and velocity from an output of an inertial sensor worn by a subject, obtain information regarding weight of the subject, and calculate an exercise output indicating an exercise amount within a predetermined time from the information regarding the acceleration, the velocity, and the weight.

According to this application example, the computer can be caused to perform control such that the information regarding the acceleration and the velocity can be acquired by the inertial sensor worn by the subject, the information regarding the weight of the subject is acquired, the exercise output indicating the exercise amount of the subject within the predetermined time is obtained, and the exercise ability of the subject is evaluated based on the obtained exercise output.

### Application Example 29

This application example is directed to the program according to the application example described above, which further causes the computer to measure a pulse rate of the subject by a pulsimeter worn by the subject and calculate a cardiac output based on the pulse rate and the exercise output.

According to this application example, the computer can be caused to perform control such that the cardiac output is calculated based on the pulse rate and the exercise ability of the subject and the exercise ability of the subject is obtained using the cardiac output.

### BRIEF DESCRIPTION OF THE DRAWINGS

The invention will be described with reference to the accompanying drawings, wherein like numbers reference like elements.

FIG. 1 is a diagram for describing an overview of an exercise ability evaluation system according to a first embodiment.

FIG. 2 is a functional block diagram illustrating the configuration of the exercise ability evaluation system according to the first embodiment.

FIG. 3 is a flowchart illustrating an example of a procedure of an exercise ability evaluation process.

FIG. 4 is a diagram for describing an example of an exercise ability evaluation data generation process and is a diagram for describing an overview of a comparison process of comparing measurement data to reference data.

FIG. 5 is a diagram for describing an overview of a process of generating the exercise ability evaluation data through the comparison process of FIG. 4.

FIG. 6 is a diagram for describing an overview of an exercise ability calculation system according to a fifth embodiment.

FIG. 7 is a functional block diagram illustrating examples of the configurations of an exercise ability calculation apparatus and a display apparatus according to the fifth embodiment.

FIG. 8 is a flowchart illustrating an example of a procedure of an exercise ability calculation process.

FIG. 9 is a diagram for describing an example of evaluation of an exercise ability of a user in an exercise ability calculation process.

FIG. 10 is a diagram for describing an example of evaluation of an exercise ability of a user in an exercise ability calculation process of a sixth embodiment.

FIG. 11 is a diagram for describing a method of obtaining a relation between a pulse rate and an exercise output of an estimation section from a relation between a pulse rate and an exercise output of an actual measurement section.

FIG. 12 is a diagram for describing a method of calculating an exercise ability of a subject from an estimation result illustrated in FIG. 11.

### DESCRIPTION OF EXEMPLARY EMBODIMENTS

Hereinafter, embodiment of the invention will be described with reference to the drawings. In the following descriptions, layers or members are illustrated at scales different from actual scales of layers or members so that the sizes of the layers or members can be recognized.

All of the constituent to be described below may not necessarily be essential constituent requisites of the invention.

### First Embodiment

First, a schematic configuration of an exercise ability evaluation system according to a first embodiment will be described with reference to the drawings.

### 1. Exercise Ability Evaluation System

### 1. Overview of System

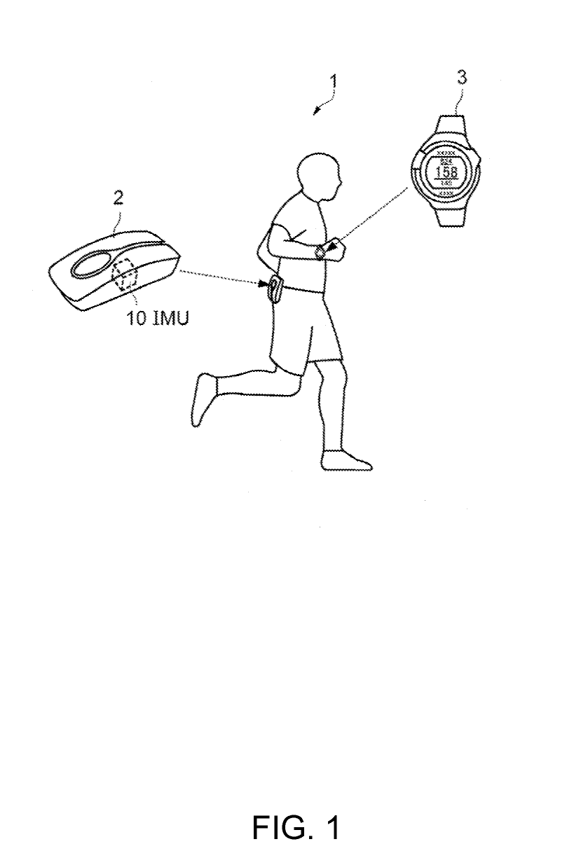

FIG. 1 is a diagram for describing an overview of an exercise ability evaluation system 1 according to the embodiment. As illustrated in FIG. 1, the exercise ability evaluation system 1 according to the embodiment is configured to include an exercise ability evaluation apparatus 2 and a display apparatus 3.

The exercise ability evaluation apparatus 2 according to the embodiment is worn on a trunk part (for example, a right waist, a left waist, or a middle part of the waist) of a user. The exercise ability evaluation apparatus 2 includes an inertial measurement unit (IMU) 10 as a measurement device, comprehends a motion during running of the user (also including walking), calculates a velocity, a position, posture angles (a roll angle, a pitch angle, and a yaw angle), and the like, further analyzes an exercise of the user, and measures a physical amount of whole body endurance of the user.

In the embodiment, when the user stops, the exercise ability evaluation apparatus 2 is worn by the user so that one detection axis (hereinafter assumed to be the z axis) of the inertial measurement unit (IMU) 10 substantially matches a gravitational acceleration direction (vertical downward direction). The exercise ability evaluation apparatus 2 transmits at least some of the exercise ability evaluation information generated based on measurement data of the measured physical amounts of the whole body endurance of the user to the display apparatus 3.

The display apparatus 3 is a wrist type (wristwatch type) portable information apparatus and is worn on a wrist or the like of the user. Here, the display apparatus 3 may be a portable information apparatus such as a head mount display (HMD) or a smartphone. The user can operate the display apparatus 3 before start of running or during the running and give an instruction to start measurement (an inertial navigation calculation process and an exercise ability evaluation process to be described below) by the exercise ability evaluation apparatus 2 (perform measurement start) or stops the measurement (perform measurement stop).

The user can operate the display apparatus 3 after end of the running and give an instruction to start or end feedback information display (to be described below) or a running analysis process (to be described below) based on exercise ability evaluation information by the exercise ability evaluation apparatus 2. The display apparatus 3 transmits a command to give an instruction of the measurement start or the measurement stop, a command to give an instruction to start or end the feedback information display or the running analysis process based on the exercise ability evaluation information, and the like to the exercise ability evaluation apparatus 2.

When the exercise ability evaluation apparatus 2 receives the command of the measurement start, the exercise ability evaluation apparatus 2 starts measurement by the inertial measurement unit (IMU) 10, analyzes an exercise ability (muscle ability) of the user based on a result of comparison and evaluation between the measurement data and reference data to be described below, and generates exercise ability evaluation information. The exercise ability evaluation apparatus 2 transmits at least some of the generated exercise ability evaluation information to the display apparatus 3. Then, the display apparatus 3 receives the exercise ability evaluation information and presents the received exercise ability evaluation information to the user in various forms such as text, figures, sound, and vibration. The user can recognize the exercise ability evaluation information (feedback information and an advise) via the display apparatus 3 during the running.

When the exercise ability evaluation apparatus 2 receives the command to give the instruction to start a running analysis process, the exercise ability evaluation apparatus 2 analyzes previous running using exercise analysis information generated during previous running and transmits information regarding an analysis result to the display apparatus 3. Then, the display apparatus 3 receives the information regarding the analysis result and presents the received exercise analysis information to the user in various forms such as text, figures, sound, and vibration. The user can recognize the analysis result of the previous running via the display apparatus 3. An information apparatus such as a personal computer can also be used instead of the display apparatus 3.

Data communication between the exercise ability evaluation apparatus 2 and the display apparatus 3 may be wireless communication or may be wired communication.

In the exercise ability evaluation system 1 according to the embodiment, a case will be exemplified in detail in which the exercise ability evaluation apparatus 2 generates the exercise ability evaluation information based on data of the whole body endurance at the time of a running exercise (running) of the user, but the invention is not limited thereto. The same can also apply even when the exercise ability evaluation information is generated based on data of the whole body endurance in an exercise other than the running exercise.

### 2. Coordinate System

Coordinate systems necessary in the following description are defined as follows:
an e frame (Earth Centered Earth Fixed Frame): a 3-dimensional rectangular coordinate system of a right-handed type in which the center of the Earth is set to an origin and the z axis is formed in parallel to the rotational axis of the earth;
an n frame (Navigation Frame): a 3-dimensional rectangular coordinate system in which a moving object (user) is set to an origin and the x, y, and z axes are set as the north, the east, and the direction of gravity, respectively;
a b frame (Body Frame): a 3-dimensional rectangular coordinate system in which a sensor (the inertial measurement unit (IMU) 10) servers as a reference; and
an m frame (Moving Frame): a 3-dimensional rectangular coordinate system of a right-handed type in which a moving object (user) is set to an origin and a travel direction of the moving object (user) is set to the x direction.

### 3. Configuration of System

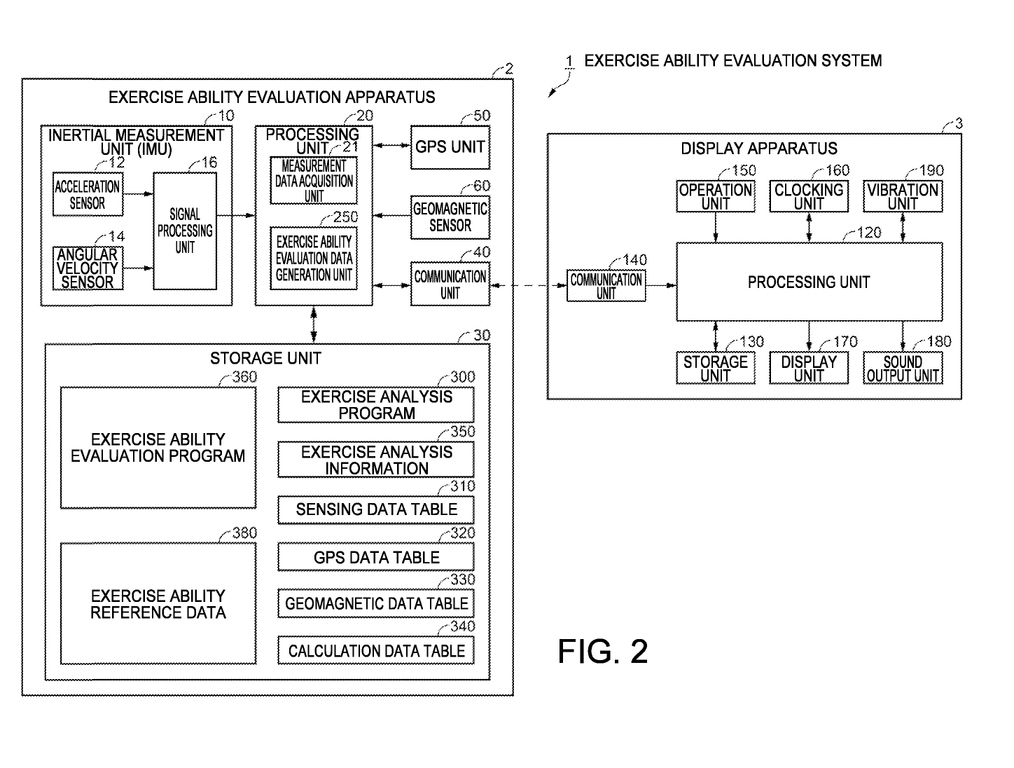

FIG. 2 is a functional block diagram illustrating the configuration of the exercise ability evaluation system 1 according to the embodiment. As illustrated in FIG. 2, the exercise ability evaluation apparatus 2 of the exercise ability evaluation system 1 is configured to include an inertial measurement unit (IMU) 10, a processing unit 20, a storage unit 30, a communication unit 40, a Global Positioning System (GPS) unit 50, and a geomagnetic sensor 60.

In the exercise ability evaluation apparatus 2 according to the embodiment, some of these constituent elements may be deleted or modified or other constituent elements may be added.

The inertial measurement unit 10 (which is an example of an inertial sensor) is configured to include an acceleration sensor 12, an angular velocity sensor 14, and a signal processing unit 16.

The acceleration sensor 12 detects an acceleration in each of the three axes directions intersecting each other (ideally crossing at right angles) and outputs a digital signal (acceleration data) according to the magnitude and direction of the detected three-axes acceleration.

The angular velocity sensor 14 detects an angular velocity in each of the three axes directions intersecting each other (ideally crossing at right angles) and outputs a digital signal (angular velocity data) according to the magnitude and direction of the detected three-axes angular velocity.

The signal processing unit 16 receives the acceleration data and the angular velocity data from each of the acceleration sensor 12 and the angular velocity sensor 14, adds time information, and stores the acceleration data and the angular velocity data in a storage unit (not illustrated). The stored acceleration data and angular velocity data and the time information are generated as sensing data in accordance with a predetermined format and are output to the processing unit 20.

The acceleration sensor 12 and the angular velocity sensor 14 are mounted so that the three axes matches the three axes of the sensor coordinate system (the b frame) using the inertial measurement unit 10 as a reference. When a mounting angle error occurs between the three axes and the three axes of the sensor coordinate system (the b frame) at the time of the mounting of the acceleration sensor 12 and the angular velocity sensor 14, a conversion process is performed by the signal processing unit 16. Specifically, the signal processing unit 16 performs a process of converting the acceleration data and the angular velocity data into data of the sensor coordinate system (the b frame) using a correction parameter calculated in advance according to the mounting angle error. Instead of the signal processing unit 16, the processing unit 20 to be described below may perform the conversion process.

The signal processing unit 16 may perform a temperature correction process of the acceleration sensor 12 and the angular velocity sensor 14. Instead of the signal processing unit 16, the processing unit 20 to be described below may perform the temperature correction process or a temperature correction function may be embedded in the acceleration sensor 12 and the angular velocity sensor 14.

The acceleration sensor 12 and the angular velocity sensor 14 may output analog signals. In this case, the signal processing unit 16 may perform A/D conversion on an output signal of the acceleration sensor 12 and an output signal of the angular velocity sensor 14 to generate sensing data.

The GPS unit 50 receives a GPS satellite signal transmitted from a GPS satellite which is a kind of positioning satellite, performs positioning calculation using the GPS satellite signal to calculate the position and the velocity (a vector including a magnitude and a direction) of the user in the n frame, and outputs GPS data to which time information or positioning accuracy information is granted to the processing unit 20. Since a method of calculating the position or the velocity using a GPS or a method of generating the time information is known, the detailed description will be omitted.

The geomagnetic sensor 60 detects a geomagnetism in each of the three-axes directions intersecting each other (ideally crossing at right angles) and outputs digital signal (geomagnetic data) according to the magnitude and the direction of the detected geomagnetism in the three axes to the processing unit 20.

Here, the geomagnetic sensor 60 may output an analog signal. In this case, the processing unit 20 may perform A/D conversion on the output signal of the geomagnetic sensor 60 to generate geomagnetic data.

The storage unit 30 is configured to include any of various IC memories such as a read-only memory (ROM), a flash ROM, and a random access memory (RAM) or a recording medium such as a hard disk or a memory card.

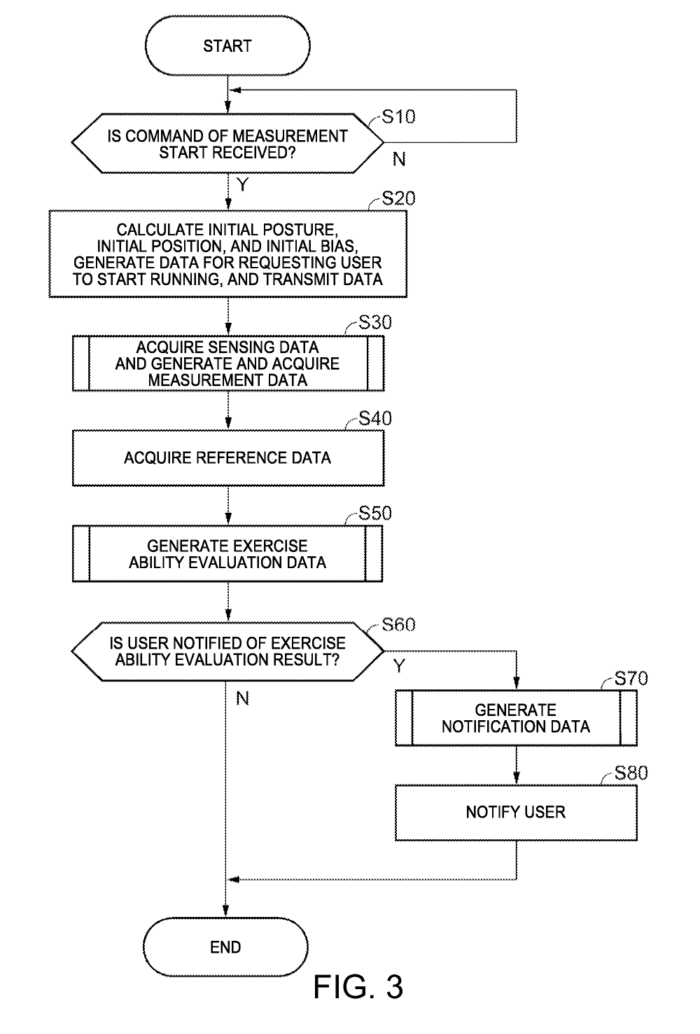

The storage unit 30 stores an exercise ability evaluation program 360 that is read by the processing unit 20 to execute an exercise ability evaluation process (see FIG. 3) and an exercise analysis program 300 that executes an exercise analysis process.

The storage unit 30 further stores, for example, an exercise ability reference data 380 serving as reference data, a sensing data table 310, a GPS data table 320, a geomagnetic data table 330, a calculation data table 340, and exercise analysis information 350.

The exercise ability reference data 380 is reference data of whole body endurance. The exercise ability reference data 380 is compared to measurement data of the whole body endurance of the user acquired by the measurement data acquisition unit 21 to be described below when the processing unit 20 generates exercise ability evaluation information. In the embodiment, a maximum oxygen intake amount is used as an index of the whole body endurance and a reference value of the maximum oxygen intake amount of every age is stored as the exercise ability reference data 380 in the storage unit 30 according to the men and women.

The sensing data table 310 is a data table that chronologically stores sensing data (detection results of the inertial measurement unit 10) received from the inertial measurement unit 10 by the processing unit 20.

The GPS data table 320 is a data table that chronologically stores GPS data (detection results of the GPS unit (GPS sensor) 50) received from the GPS unit 50 by the processing unit 20.

The geomagnetic data table 330 is a data table that chronologically stores geomagnetic data (detection results of the geomagnetic sensor) received from the geomagnetic sensor 60 by the processing unit 20.

The calculation data table 340 is a data table that chronologically stores velocities, positions, and posture angles calculated using the sensing data received from the inertial measurement unit 10 by the processing unit 20.

The exercise analysis information 350 is various kinds of information regarding an exercise of the user and includes various kinds of information such as analysis information or running trajectory information of the exercise of the user generated by the processing unit 20 and input information regarding a weight or the like input in advance by the user.

The processing unit 20 includes a measurement data acquisition unit 21 and an exercise ability evaluation data generation unit 250 serving as an exercise ability evaluation information generation unit, is configured by, for example, a central processing unit (CPU), a digital signal processor (DSP), or an application specific integrated circuit (ASIC), and performs various calculation processes or control processes according to various programs stored in the storage unit 30.

The measurement data acquisition unit 21 receives the sensing data, the GPS data, and the geomagnetic data from the inertial measurement unit 10, the GPS unit 50, and the geomagnetic sensor 60, respectively, and calculates the velocity, the position, the posture angle, and the like of the user using the data to acquire the velocity, the position, the posture angle, and the like of the user as measurement data of the whole body endurance of the user.

The processing unit 20 includes the exercise ability evaluation data generation unit 250 as an exercise ability evaluation information generation unit. The exercise ability evaluation data generation unit 250 compares the measurement data of the measurement data acquisition unit 21 to the exercise ability reference data 380 of the storage unit 30 and generates exercise ability evaluation information in which a muscle ability of the user is a main ability based on the comparison result.

Then, the processing unit 20 transmits at least some of the pieces of generated exercise ability evaluation information to the display apparatus 3 via the communication unit 40. The display apparatus 3 outputs the received exercise ability evaluation information in forms such as text, images, sound, and vibration.

The communication unit 40 performs data communication with a communication unit 140 of the display apparatus 3 and performs, for example, a process of receiving the exercise ability evaluation information generated by the processing unit 20, output information during running, or output information after running and transmitting the received information to the display apparatus 3 or a process of receiving a command (a command of the measurement start/the measurement stop, a command to start/end the running analysis process, or the like) transmitted from the display apparatus 3 and transmitting the command to the processing unit 20.

The display apparatus 3 is configured to include a processing unit 120, a storage unit 130, the communication unit 140, an operation unit 150, a clocking unit 160, a display unit 170, a sound output unit 180, and a vibration unit 190. However, the display apparatus 3 according to the embodiment may be configured such that some of these constituent elements may be deleted or modified or other constituent elements may be added.

The processing unit 120 performs various calculation processes or control processes according to programs stored in the storage unit 130. Specifically, the processing unit 120 performs various processes (for example, a process of transmitting the command of the measurement start/the measurement stop or the command to start/end the running analysis process to the communication unit 140, a display process according to the operation data, and a sound output process) according to operation data received from the operation unit 150. The processing unit 120 performs a process of receiving the output information during running or the output information after running from the communication unit 140 and sending text data or image data according to the output information during running or the output information after running to the display unit 170. The processing unit 120 further performs a process of transmitting sound data according to the output information during running or the output information after running to the sound output unit 180 or a process of transmitting vibration data according to the output information during running to the vibration unit 190. The processing unit 120 further performs, for example, a process of generating time image data according to time information received from the clocking unit 160 and transmitting the time image data to the display unit 170.

For example, the storage unit 130 is configured by any of various IC memories such as a ROM storing data or programs used for the processing unit 120 to perform various processes or a RAM serving as a work area of the processing unit 120.

The communication unit 140 performs data communication with the communication unit 40 of the exercise ability evaluation apparatus 2. Specifically, the communication unit 140 performs a process of receiving a command (for example, a command of the measurement start/the measurement stop or a command to start/end the running analysis process) according to operation data from the processing unit 120 and transmitting the command to the exercise ability evaluation apparatus 2. The communication unit 140 further performs a process of receiving the output information during running, the output information after running, or the exercise ability evaluation information transmitted from the exercise ability evaluation apparatus 2 and transmitting the received information to the processing unit 120.

The operation unit 150 performs a process of acquiring operation data (operation data of the measurement start/the measurement stop, selection of display content, or the like) from the user and transmitting the operation data to the processing unit 120. The operation unit 150 may be, for example, a touch panel type display, a button, a key, or a microphone.

The clocking unit 160 performs a process of generating time information of year, month, day, hour, minute, second, or the like. For example, the clocking unit 160 is realized by a real time clock (RTC) IC or the like.

The display unit 170 displays image data or text data transmitted from the processing unit 120 as text, a graph, a table, animation, or another image. For example, the display unit 170 may be realized by a display such as a liquid crystal display (LCD), an organic electroluminescence (EL) display, an electrophoretic display (EPD) and may be a touch panel type display. The functions of the operation unit 150 and the display unit 170 may be realized by one touch panel type display.

The sound output unit 180 outputs the sound data transmitted from the processing unit 120 as sound such as voice or buzzer sound. For example, the sound output unit 180 is realized by a speaker, a buzzer, or the like.

The vibration unit 190 vibrates according to the vibration data transmitted from the processing unit 120. The vibration is delivered to the display apparatus 3 so that the user wearing the display apparatus 3 can feel the vibration. For example, the vibration unit 190 is realized by a vibration motor or the like.

### Exercise Ability Evaluation Method (Procedure of Process)

Next, an exercise ability evaluation method for the user will be described with reference to the drawings. FIG. 3 is a flowchart illustrating an example of a procedure of the exercise ability evaluation process (which is an example of an exercise ability evaluation method) performed by the processing unit 20. The processing unit 20 performs the exercise ability evaluation process in the procedure of the flowchart of FIG. 3 by executing the exercise ability evaluation program 360 stored in the storage unit 30.

In the exercise ability evaluation method according to the embodiment, the measurement data of the whole body endurance at the time of exercise of the user is acquired, the measurement data is compared to the reference data stored in the exercise ability reference data 380 of the storage unit 30, and the muscle ability of the user is evaluated based on the comparison result, and the exercise ability evaluation information is generated. In the embodiment, a maximum oxygen intake amount VO₂ₘₐₓ is used as an index for calculating the whole body endurance of the user and a running distance in which the user runs with the utmost effort per unit time is used as the evaluation data for estimating the maximum oxygen intake amount VO₂ₘₐₓ. As the value of the maximum oxygen intake amount VO₂ₘₐₓ is higher, the an energy generation amount increases. Thus, since the user do an exercise of a high exercise intensity, the maximum oxygen intake amount VO₂ₘₐₓ can be used properly as an index of the whole body endurance originated from the muscle ability. In the embodiment, an example in which a male user performs the exercise ability evaluation method will be described.

In FIG. 3, the processing unit 20 waits until the command of the measurement start is received (N in step S10). When the command of the measurement start is received (Y in step S10), the processing unit 20 calculates an initial posture, an initial position, and an initial bias using the sensing data and the GPS data measured by the inertial measurement unit 10 on the assumption that the user stops, generates data for requesting the user to start running with his or her utmost effort, and transmits the data to the display apparatus 3 (step S20).

Of the text data, the image data, the sound data, and the vibration data above described, the data for requesting the user to start the running is preferably the sound data or the vibration data by which the user can recognize that the user is requested to start the running even when the user does not continuously watch the display unit 170. However, the text data or the image data may be displayed by the display apparatus 3.

The user starts the running with the utmost effort when the user recognizes the data for requesting the user to start the running through sound, vibration, or the like from the display apparatus 3.

After a predetermined time has passed from the running start of the user in step S20, the processing unit 20 acquires the sensing data from the inertial measurement unit 10 and adds the acquired sensing data to the sensing data table 310 (step S30). In the embodiment, as described above, the running distance of the user per unit time is set as the evaluation data for estimating the maximum oxygen intake amount VO₂ₘₐₓ. Therefore, in step S30, the running distance by the running of the user with the utmost effort per unit time is calculated from the initial position of the user calculated in step S20 and the position of the user after the elapse of a predetermined time. The calculated running distance is acquired as the measurement data by the measurement data acquisition unit 21.

Next, the processing unit 20 acquires the reference data used for comparison evaluation with the measurement data from the exercise ability reference data 380 of the storage unit 30 (step S40). An example of the reference data acquired from the exercise ability reference data 380 will be described below.

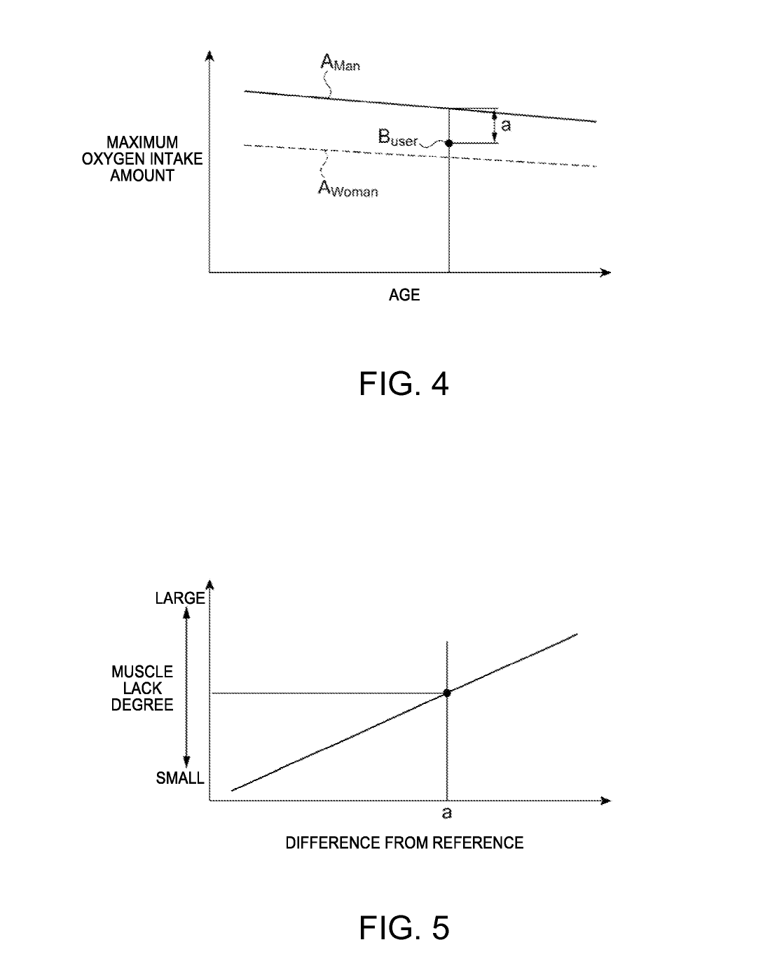

Next, the processing unit 20 compares the measurement data acquired in step S30 by the measurement data acquisition unit 21 to the reference data acquired in step S40 from the exercise ability reference data 380 and performs control such that the exercise ability evaluation data generation unit 250 generates the exercise ability evaluation data for which the muscle ability of the user is the main ability (step S50). Hereinafter, an example of the exercise ability evaluation data generation process will be described with reference to the drawing. FIG. 4 is a diagram for describing an example of the exercise ability evaluation data generation process and is a diagram for describing an overview of a comparison process of comparing the measurement data to the reference data. FIG. 5 is a diagram for describing an overview of a process of generating the exercise ability evaluation data through the comparison process of FIG. 4.

In FIG. 4, the vertical axis represents the maximum oxygen intake amount VO₂ₘₐₓ which is an index of the whole body endurance according to the embodiment and the horizontal axis represents an age. In a graph indicating a relation of the two-axes index, reference data Aₘₐₙ indicating reference values for men of every age of the maximum oxygen intake amount VO₂ₘₐₓ and reference data A_{woman} indicating reference values for women of every age of the maximum oxygen intake amount VO₂ₘₐₓ are plotted.

In FIG. 4, an actual measurement value B_{User} of the maximum oxygen intake amount VO₂ₘₐₓ of the user estimated from the actual measurement data (actually measured values) of the running distance obtained in the running of the user with the utmost effort per unit time is plotted. As a method of estimating the maximum oxygen intake amount from the running distance obtained in the running with the utmost effort per unit time, a known method according to the run unit time can be used. For example, on the assumption that a distance by which the user runs as “12-minute running” is X(m) when the run unit time is 12 minutes, the maximum oxygen intake amount VO₂ₘₐₓ can be estimated by the following estimation expression.

On the assumption that a distance by which the user runs as “15-minute running” is X(m) when the run unit time is 15 minutes, the maximum oxygen intake amount VO₂ₘₐₓ can be estimated by the following estimation expression.

The reference data A_{Man} indicating reference values of men of every age of the maximum oxygen intake amount VO₂ₘₐₓ and reference data A_{Woman} indicating reference values of women of every age of the maximum oxygen intake amount VO₂ₘₐₓ may be generated from the running distance X(m) of the above-described 12-minute running or 15-minute running using the estimation expression (Expression 1) or the estimation expression (Expression 2), and may be stored in the exercise ability reference data 380 of the storage unit 30.

In FIG. 4, the maximum oxygen intake amount VO₂ₘₐₓ of the user which is a man is smaller by a than the reference value of the maximum oxygen intake amount VO₂ₘₐₓ of a man of the same age. This difference indicates the degree when the whole body endurance originated from the muscle ability (muscle amount) is smaller than the reference value of the same age. An overview of an example of the process of generating the exercise ability evaluation data through the comparison process of comparing the actual measurement value (the measurement data) to the reference data is illustrated in the explanatory diagram of FIG. 5. In FIG. 5, the horizontal axis represents the magnitude of a difference between the actual measurement value of the maximum oxygen intake amount VO₂ₘₐₓ of the user and the reference value of the same age and the horizontal axis represents a muscle lack degree (a muscle lack degree with respect to the reference value) estimated from the magnitude of the difference. In FIG. 5, a which is the magnitude of the difference from the reference value of the maximum oxygen intake amount VO₂ₘₐₓ described in FIG. 4 is illustrated for description. As illustrated in FIG. 5, of course, as the difference between the actual measurement value of the maximum oxygen intake amount VO₂ₘₐₓ and the reference value of the same age is larger, the muscle lack degree is larger. The exercise ability evaluation data generation unit 250 generates the muscle lack degree or the like as the exercise ability evaluation data.

The age is used on the horizontal axis of FIG. 4, but another index may be used solely or compositely on the horizontal axis or may be used as an element correcting the index of the age or the like of FIG. 4. For example, an index such as a weight, a body composition, a lactic acid value associated with an exercise, a maximum heart rate, or a relative value of an everyday exercise level may be used solely or compositely on the horizontal axis of FIG. 4 or may be used as a coefficient. Thus, accuracy of the generated exercise ability evaluation data can be improved.

Next, the processing unit 20 determines whether the user is notified of the content of the exercise ability evaluation data generated in step S50 (step S60). When the processing unit 20 determines whether it is not necessary to notify the user of the content of the generated exercise ability evaluation data (N in step S60), the exercise ability evaluation data generation process ends. Apart from this, when the processing unit 20 determines that the user is notified of the generated exercise ability evaluation data, for example, when the muscle lack degree illustrated in FIG. 5 exceeds the threshold value, (Y in step S60), the processing unit 20 generates notification data (advice data) from a kind of desired data of which the user is notified, based on the content of the exercise ability evaluation data (step S70) and transmits the notification data to the display apparatus 3 via the communication unit 40 (step S80). As the notification data, for example, various kinds of notification are considered, such as messages used to notify the user that the muscle lack degree is greater than that of the same sex of the same age and that training for the whole body endurance including a muscular strength is necessary.

The user may be normally notified of the content of the generated exercise ability evaluation data in real time.

After the user is notified of the notification data, a series of exercise ability evaluation data generation processes ends.

As described above, in the exercise ability evaluation system 1 and the exercise ability evaluation method using the exercise ability evaluation system 1 according to the embodiment, a relative evaluation result of the whole body endurance originated from the muscle ability of the user can be denoted for the user. Thus, for example, it is possible to expect advantages of denoting superiority or inferiority of the muscle ability of the user or the degree of the muscle ability of the user with respect to the reference value of the muscle ability of the same sex and the same age as the user and causing the user to recognize the superiority or inferiority or the degree, and thus giving a hint to the user who decides a subsequent training method or achieving an improvement in a motivation for the training of the user.

### OTHER EMBODIMENTS

The invention is not limited to the above-described first embodiment, but modifications, improvements, or the like of the above-described embodiment can be made. Other embodiments of the foregoing embodiment will be described below.

### Second Embodiment

As the exercise ability evaluation method according to the first embodiment, the method of calculating the maximum oxygen intake amount VO₂ₘₐₓ as the actual measurement value of the running distance such as 12-minute running or 15-minute running using the maximum oxygen intake amount VO₂ₘₐₓ in the index of the whole body endurance of the user and substituting the running distance to the estimation expression has been described.

The invention is not limited thereto. The actual measurement value of the maximum oxygen intake amount VO₂ₘₐₓ of the user can be calculated through a “shuttle run test.”

Of the shuttle run tests, in particular, in a general “20 m shuttle run” measurement method, a user performs a shuttle run of distance 20 m (round-trip endurance run) for 2 minutes at a speed of 8 km per hour, and then gradually increase a pace rhythm of every 0.5 km per hour for every 2 minutes. When the user may not follow the pace rhythm, an individual maximum running speed is assumed to be X [km/h]. The maximum oxygen intake amount VO₂ₘₐₓ when the maximum running speed is X [km/h] can be calculated with the following estimation expression.

In the exercise ability evaluation method according to the second embodiment, the running of the user is performed such that the user approaches the utmost effort step by step by a gradually increasing load. Therefore, since a hard time in which the utmost effort is exerted is a short time compared to the first embodiment in which the user runs with his or her utmost effort for the decided time such as 12 minutes or 15 minutes, there is an advantage in which the work is relatively safe for the user.

### Third Embodiment

In the exercise ability evaluation method according to the first embodiment, the maximum oxygen intake amount VO₂ₘₐₓ has been used as the index of the whole body endurance of the user, and the maximum oxygen intake amount VO₂ₘₐₓ has been obtained by substituting the running distance such as 12-minute running or 15-minute running in which the user runs with the utmost effort per unit time as the actual measurement value (measurement data) to the estimation expression.

The invention is not limited thereto. For example, a necessary time from a start to a goal in which the user runs a full-marathon or a half-marathon of a predetermined distance with the utmost effort may be set as the measurement data (actual measurement value).

In the exercise ability evaluation method according to the third embodiment, it is possible to expect an advantage of obtaining exercise ability evaluation data of close content in sports for a marathon runner, a user continuously doing an exercise with a relatively high exercise intensity, or users doing the same sports or of improving a motivation.

### Fourth Embodiment

In the first embodiment, in the exercise ability evaluation method using the maximum oxygen intake amount VO₂ₘₐₓ as the index of the whole body endurance of the user, the exercise ability evaluation data has been generated by detecting positional information of the user in the measurement data acquisition work (the running) to measure the running distance by the acceleration sensor 12 and the angular velocity sensor 14 of the inertial measurement unit (IMU) 10, the GPS unit 50, and the like worn by the user and estimating the maximum oxygen intake amount. The exercise ability evaluation data of the user based on the comparison result with the reference data has been generated by setting the running distance in the running of the utmost effort of the user per unit time as the actual measurement value (measurement data).

The invention is not limited thereto, but a plurality of sensors can be employed and the maximum oxygen intake amount VO₂ₘₐₓ can be obtained from measurement data obtained by measuring an exercise output, biological information, or the like as well as the positional information of the user who is doing an exercise. Hereinafter, an embodiment of this method will be described.

The acceleration sensor 12 and the angular velocity sensor 14 of the inertial measurement unit (IMU) 10, the GPS unit 50, and the like worn by the user can measure an exercise output (body movement data) of the user who is moving as well as the positional information of the user who is running.

In the embodiment, the user wears a pulse sensor (not illustrated) that measures a pulse of the user who is running in a running test. Then, an oxygen intake amount (the maximum oxygen intake amount VO₂ₘₐₓ) can be calculated based on a pulse and an exercise output obtained from pulse data of the user obtained by the pulse sensor during the running test and an exercise output of the user measured by the inertial measurement unit 10.

Here, for example, when the user wears a wristwatch type pulsimeter mounted on the display apparatus 3 in FIG. 1 or winds a Hartley sensor around his or her chest with a belt and runs, the pulsimeter worn by the user may calculate a heart rate during the running of the user as a first item of exercise analysis information using a measurement value of the pulsimeter or the Hartley sensor.

A heart rate (HRᵣₑₛₜ) at the time of rest and a maximum heart rate (HRₘₐₓ) can be obtained from the above-described pulse data, and a relative value of the maximum oxygen intake amount (VO₂ₘₐₓ) can be further estimated from the heart rate (HRᵣₑₛₜ) at the time of rest and the maximum heart rate (HRₘₐₓ) by the following estimation expression.

In the method of estimating the maximum oxygen intake amount according to the embodiment, the maximum oxygen intake amount can be obtained relatively easily without causing the user to do an unreasonable work (exercise) when the maximum heart rate and the heart rate at the time of rest can be obtained.

The embodiment of the invention devised by the inventors has been described specifically above, but the invention is not limited to the foregoing embodiment and can be modified in various ways within the scope of the invention without departing from the gist of the invention.

For example, in the exercise ability evaluation system 1 according to the foregoing embodiments, the inertial measurement unit (IMU) 10 serving as a measurement device is configured to be included in the exercise ability evaluation apparatus 2 and the exercise ability evaluation apparatus 2 is configured to be worn on a trunk part of the user. However, the invention is not limited thereto. The inertial measurement unit 10 may be included as a unit distinguished from the exercise ability evaluation apparatus 2 and may be configured to be worn on a trunk part of the user by a different wearing mechanism from the exercise ability evaluation apparatus 2. Thus, since the inertial measurement unit (IMU) 10 which is a measurement device can be miniaturized, it is possible to further obtain advantages of improving the degree of freedom of a worn position and preventing an exercise of the user from being interrupted.

In the foregoing embodiments, the maximum oxygen intake amount VO₂ₘₐₓ has been configured to be used as the index of the whole body endurance of the user, and the maximum oxygen intake amount VO₂ₘₐₓ has been configured to be estimated using the running distance such as 12-minute running or 15-minute running in which the user runs with the utmost effort per unit time as the actual measurement value (measurement data).

The invention is not limited thereto. For example, the whole body endurance of the user originated from the muscle ability of the user can be relatively comprehended using a necessary time from a start to a goal in which the user runs a full-marathon or a half-marathon of a predetermined distance with the utmost effort as the measurement data (actual measurement value), and then the exercise ability evaluation data can also be generated.

In the foregoing embodiments, the example has been described in which the reference data when the oxygen intake amount is an evaluation axis of the whole body endurance is estimated by the estimation expression. However, an actual measurement value measured by an expiration gas measurement instrument may, of course, be used.

In the foregoing embodiments, the example has been described in which the maximum oxygen intake amount is used as the index of the whole body endurance. However, the exercise ability evaluation data can be generated using a different index of the whole body endurance from the maximum oxygen intake amount.

Examples of the different index of the whole body endurance from the maximum oxygen intake amount include an oxygen intake amount (VO), a cardiorespiratory ability, a lactic acid value, and a degree of fatigue. Measurement data of the whole body endurance can be acquired as this index and the exercise ability evaluation data can be generated.

In the foregoing embodiments, the estimation expressions (Expression 1) to (Expression 4) obtaining the maximum oxygen intake amount which is one of the indexes of the whole body endurance have been described. However, coefficients in such expressions are representative examples and the invention is not limited thereto.

### Fifth Embodiment

First, a schematic configuration of an exercise ability calculation system according to a fifth embodiment will be described with reference to the drawings.

### 1. Exercise Ability Calculation System

### 1. Overview of System

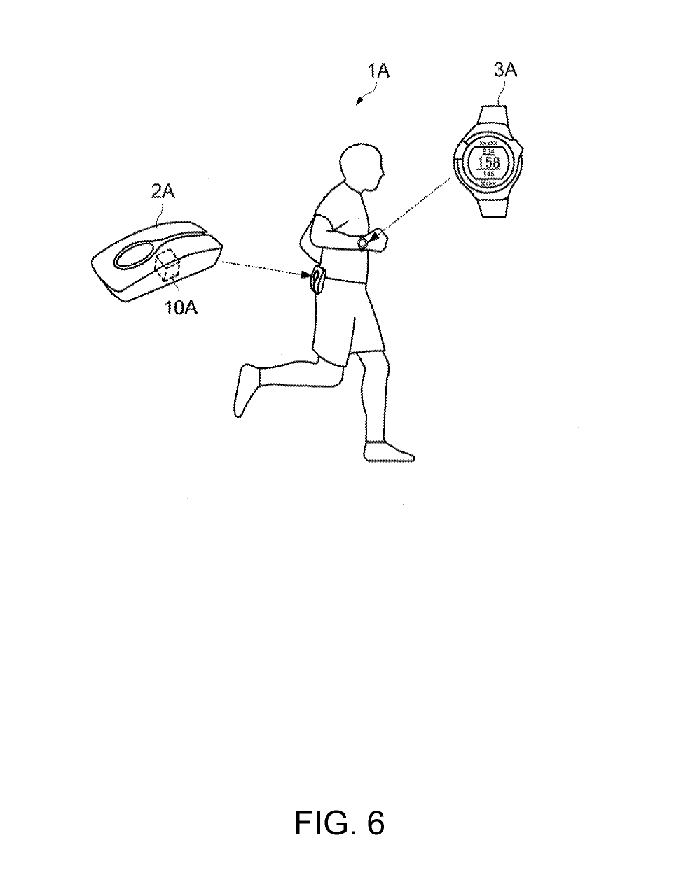

FIG. 6 is a diagram for describing an overview of an exercise ability calculation system 1A according to the embodiment. As illustrated in FIG. 6, the exercise ability calculation system 1A according to the embodiment is configured to include an exercise ability calculation apparatus 2A and a display apparatus 3A.

The exercise ability calculation apparatus 2A according to the embodiment is worn on a trunk part (for example, a right waist, a left waist, or a middle part of the waist) of a subject. The exercise ability calculation apparatus 2A includes an inertial measurement unit (IMU) 10A including an inertial sensor, comprehends a motion during running of the subject (also including walking), calculates a velocity, a position, posture angles (a roll angle, a pitch angle, and a yaw angle), and the like, further analyzes an exercise of the subject, and measures a physical amount of whole body endurance of the subject. In the embodiment, when the subject stops, the exercise ability calculation apparatus 2A is worn by the subject so that one detection axis (hereinafter assumed to be the z axis) of the inertial measurement unit (IMU) 10A substantially matches a gravitational acceleration direction (vertical downward direction).

The display apparatus 3A is a wrist type (wristwatch type) portable information apparatus and is worn on a wrist or the like of the subject. Here, the display apparatus 3A may be a portable information apparatus such as a head mount display (HMD) or a smart phone. The subject can operate the display apparatus 3A before start of running or during the running and give an instruction to start measurement (an inertial navigation calculation process and an exercise ability calculation process to be described below) by the exercise ability calculation apparatus 2A (perform measurement start) or stops the measurement (perform measurement stop). The subject can operate the display apparatus 3A after end of the running and give an instruction to start or end feedback information display (to be described below) or a running analysis process (to be described below) based on exercise ability evaluation information by the exercise ability calculation apparatus 2A. The display apparatus 3A transmits a command to give an instruction of the measurement start or the measurement stop, a command to give an instruction to start or end the feedback information display or the running analysis process based on the exercise ability evaluation information, and the like to the exercise ability calculation apparatus 2A.

A pulsimeter 70 (not illustrated) is mounted on the display apparatus 3A according to the embodiment. Information regarding the pulse rate measured on the wrist part of the subject by the pulsimeter 70 included in the wrist type display apparatus 3A is transmitted to the exercise ability calculation apparatus 2A via a communication unit 140A. The pulsimeter worn by the subject is not limited to, for example, the wrist type display apparatus 3A. The subject may wind a heart-rate sensor around a chest with a belt and runs, and a heart rate during running of the subject may be calculated as one item of exercise analysis information using a measurement value of the pulsimeter or the high rate sensor. In the following embodiments including the embodiment or content of other specifications, a “pulse rate” is assumed to include a “heart rate.”

When the exercise ability calculation apparatus 2A receives the command of the measurement start, the exercise ability calculation apparatus 2A starts measurement by the inertial measurement unit (IMU) 10A. Then, the exercise ability calculation apparatus 2A calculates an exercise output indicating an exercise amount of the subject within a predetermined time based on information regarding acceleration and velocity obtained from the output of the inertial sensor of the inertial measurement unit 10A, basic information regarding the separately input weight or the like of the subject, or the like and generates exercise ability data of the subject based on the calculated exercise ability. The exercise ability calculation apparatus 2A transmits at least some of the generated exercise ability data to the display apparatus 3A. Then, the display apparatus 3A receives the exercise ability data and presents the received exercise ability data to the subject in various forms such as text, figures, sound, and vibration. The subject can recognize the exercise ability data (feedback information and advice) via the display apparatus 3A during the running.

When the exercise ability calculation apparatus 2A receives the command to give the instruction to start a running analysis process, the exercise ability calculation apparatus 2A analyzes previous running using exercise analysis information generated during previous running and transmits information regarding an analysis result to the display apparatus 3A or an information apparatus such as a personal computer or a smartphone (not illustrated). Then, the display apparatus 3A or the information apparatus receives the information regarding the analysis result and presents the received exercise analysis information to the subject in various forms such as text, figures, sound, and vibration. The subject can recognize the analysis result of the previous running via the display apparatus 3A or the information apparatus.

Data communication between the exercise ability calculation apparatus 2A and the display apparatus 3A may be wireless communication or may be wired communication.

In the embodiment, a case will be exemplified in detail below in which the exercise ability calculation apparatus 2A generates the exercise ability data based on the exercise output at the time of a running exercise (running) of the subject. However, the same can also apply even when the exercise ability calculation system 1A according to the embodiment generates the exercise ability data based on the exercise output in an exercise other than the running exercise.

### 2. Coordinate System

Coordinate systems necessary in the following description are defined as follows:
an e frame (Earth Centered Earth Fixed Frame): a 3-dimensional rectangular coordinate system of a right-handed type in which the center of the Earth is set to an origin and the z axis is formed in parallel to the rotational axis of the earth;
an n frame (Navigation Frame): a 3-dimensional rectangular coordinate system in which a moving object (subject) is set to an origin and the x, y, and z axes are set as the north, the east, and the direction of gravity, respectively;
a b frame (Body Frame): a 3-dimensional rectangular coordinate system in which a sensor (the inertial measurement unit (IMU) 10A) servers as a reference; and
an m frame (Moving Frame): a 3-dimensional rectangular coordinate system of a right-handed type in which a moving object (subject) is set to an origin and a travel direction of the moving object (subject) is set to the x direction.

### 3. Configuration of System

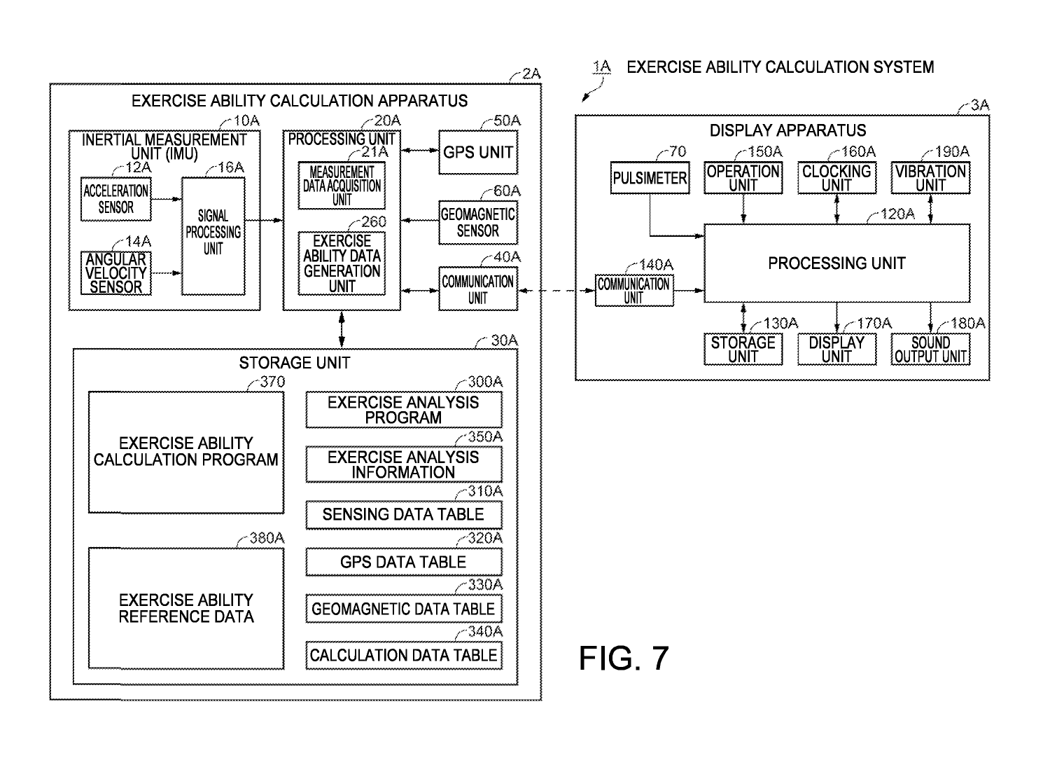

FIG. 7 is a functional block diagram illustrating configuration examples of the exercise ability calculation apparatus 2A and the display apparatus 3A. As illustrated in FIG. 7, the exercise ability calculation apparatus 2A is configured to include an inertial measurement unit (IMU) 10A, a processing unit 20A, a storage unit 30A, a communication unit 40A, a Global Positioning System (GPS) unit 50A, and a geomagnetic sensor 60A.

The processing unit 20A includes a measurement data acquisition unit 21A and an exercise ability data generation unit 260. Here, in the exercise ability calculation apparatus 2A according to the embodiment, some of these constituent elements may be deleted or modified or other constituent elements may be added.

The inertial measurement unit 10A (which is an example of an inertial sensor) is configured to include an acceleration sensor 12A, an angular velocity sensor 14A, and a signal processing unit 16A.

The acceleration sensor 12A detects an acceleration in each of the three axes directions intersecting each other (ideally crossing at right angles) and outputs a digital signal (acceleration data) according to the magnitude and direction of the detected three-axes acceleration.

The angular velocity sensor 14A detects an angular velocity in each of the three axes directions intersecting each other (ideally crossing at right angles) and outputs a digital signal (angular velocity data) according to the magnitude and direction of the detected three-axes angular velocity.

The signal processing unit 16A receives the acceleration data and the angular velocity data from each of the acceleration sensor 12A and the angular velocity sensor 14A, adds time information, and stores the acceleration data and the angular velocity data in a storage unit (not illustrated). Then, the signal processing unit 16A generates sensing data for which the stored acceleration data and angular velocity data to which the time information is attached conform to a predetermined format, and outputs the sensing data to the processing unit 20A.

The acceleration sensor 12A and the angular velocity sensor 14A are mounted so that the three axes matches the three axes of the sensor coordinate system (the b frame) using the inertial measurement unit 10A as a reference. When a mounting angle error occurs between the three axes and the three axes of the sensor coordinate system (the b frame) at the time of the mounting of the acceleration sensor 12A and the angular velocity sensor 14A, a conversion process is performed by the signal processing unit 16A. Specifically, the signal processing unit 16A performs a process of converting the acceleration data and the angular velocity data into data of the sensor coordinate system (the b frame) using a correction parameter calculated in advance according to the mounting angle error. Instead of the signal processing unit 16A, the processing unit 20A to be described below may perform the conversion process.

The signal processing unit 16A may perform a temperature correction process of the acceleration sensor 12A and the angular velocity sensor 14A. Instead of the signal processing unit 16A, the processing unit 20A to be described below may perform the temperature correction process or a temperature correction function may be embedded in the acceleration sensor 12A and the angular velocity sensor 14A.

The acceleration sensor 12A and the angular velocity sensor 14A may output analog signals. In this case, the signal processing unit 16A may perform A/D conversion on an output signal of the acceleration sensor 12A and an output signal of the angular velocity sensor 14A to generate sensing data.

The GPS unit 50A receives a GPS satellite signal transmitted from a GPS satellite which is a kind of positioning satellite, performs positioning calculation using the GPS satellite signal to calculate the position and the velocity (a vector including a magnitude and a direction) of a user which is the subject in the n frame, and outputs GPS data to which time information or positioning accuracy information is granted to the processing unit 20A. Since a method of calculating the position or the velocity using a GPS or a method of generating the time information is known, the detailed description will be omitted.

The geomagnetic sensor 60A detects a geomagnetism in each of the three-axes directions intersecting each other (ideally crossing at right angles) and outputs digital signal (geomagnetic data) according to the magnitude and the direction of the detected geomagnetism in the three axes to the processing unit 20A. Here, the geomagnetic sensor 60A may output an analog signal. In this case, the processing unit 20A may perform A/D conversion on the output signal of the geomagnetic sensor 60A to generate geomagnetic data.

The storage unit 30A is configured to include any of various IC memories such as a read-only memory (ROM), a flash ROM, and a random access memory (RAM) or a recording medium such as a hard disk or a memory card.

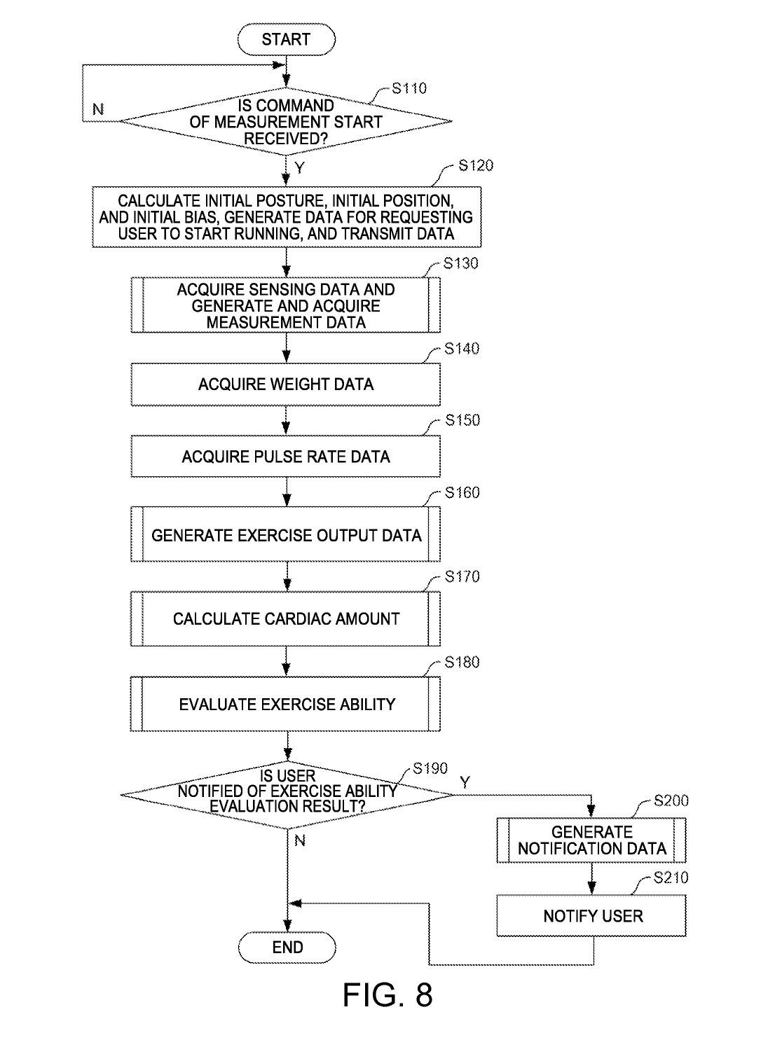

The storage unit 30A stores an exercise ability calculation program 370 that is read by the processing unit 20A serving as a calculation unit to execute an exercise ability evaluation process (see FIG. 8) and an exercise analysis program 300A that executes an exercise analysis process.

The storage unit 30A further stores, for example, an exercise ability reference data 380A serving as reference data, a sensing data table 310A, a GPS data table 320A, a geomagnetic data table 330A, a calculation data table 340A, and exercise analysis information 350A.

The exercise ability reference data 380A is reference data of whole body endurance. The exercise ability reference data 380A is compared to measurement data of the whole body endurance of the user acquired by the measurement data acquisition unit 21A to be described below when the processing unit 20A generates exercise ability evaluation information. In the embodiment, a maximum oxygen intake amount is used as an index of the whole body endurance and a reference value of the maximum oxygen intake amount of every age is stored as the exercise ability reference data 380A in the storage unit 30A according to men and women.

The sensing data table 310A is a data table that chronologically stores sensing data (detection results of the inertial measurement unit 10A) received from the inertial measurement unit 10A by the processing unit 20A.

The GPS data table 320A is a data table that chronologically stores GPS data (detection results of the GPS unit (GPS sensor) 50A) received from the GPS unit 50A by the processing unit 20A.

The geomagnetic data table 330A is a data table that chronologically stores geomagnetic data (detection results of the geomagnetic sensor) received from the geomagnetic sensor 60A by the processing unit 20A.

The calculation data table 340A is a data table that chronologically stores velocities, positions, and posture angles calculated using the sensing data by the processing unit 20A.

The exercise analysis information 350A is various kinds of information regarding an exercise of the user and includes various kinds of information such as analysis information or running trajectory information of the exercise of the user generated by the processing unit 20A and input information regarding a weight or the like input in advance by the user.

The processing unit 20A serving as a calculation unit includes a measurement data acquisition unit 21A and an exercise ability data generation unit 260. The processing unit 20A is configured by, for example, a central processing unit (CPU), a digital signal processor (DSP), or an application specific integrated circuit (ASIC) and performs various calculation processes or control processes according to various programs stored in the storage unit 30A.

The measurement data acquisition unit 21A receives, for example, the sensing data, the GPS data, and the geomagnetic data from the inertial measurement unit 10A, the GPS unit 50A, and the geomagnetic sensor 60A, respectively, and calculates the velocity, the position, the posture angle, and the like of the user using the data to acquire the velocity, the position, the posture angle, and the like as measurement data of the whole body endurance of the user.

The exercise ability data generation unit 260 compares the measurement data of the measurement data acquisition unit 21A to the exercise ability reference data 380A of the storage unit 30A and generates exercise ability evaluation information in which the muscle ability of the user is the main based on the comparison result.

Then, the processing unit 20A transmits at least some of the pieces of generated exercise ability evaluation information generated by the exercise ability data generation unit 260 to the display apparatus 3A via the communication unit 40A.

The display apparatus 3A outputs the received exercise ability evaluation information in forms such as text, images, sound, and vibration.

The communication unit 40A performs data communication with a communication unit 140A of the display apparatus 3A. The communication unit 40A performs, for example, a process of receiving the exercise ability evaluation information generated by the processing unit 20A, output information during running, or output information after running and transmitting the received information to the display apparatus 3A or a process of receiving a command (a command of the measurement start/the measurement stop, a command to start/end the running analysis process, or the like) transmitted from the display apparatus 3A and transmitting the command to the processing unit 20A.

The display apparatus 3A is configured to include a processing unit 120A, a storage unit 130A, the communication unit 140A, an operation unit 150A, a clocking unit 160A, a display unit 170A, a sound output unit 180A, and a vibration unit 190A. However, the display apparatus 3A according to the embodiment may be configured such that some of these constituent elements may be deleted or modified or other constituent elements may be added.

The display apparatus 3A includes the pulsimeter 70.

The processing unit 120A performs various calculation processes or control processes according to programs stored in the storage unit 130A. Specifically, the processing unit 120A performs various processes (for example, a process of transmitting the command of the measurement start/the measurement stop or the command to start/end the running analysis process to the communication unit 140A, a display process according to the operation data, and a sound output process) according to operation data received from the operation unit 150A. The processing unit 120A performs a process of receiving the output information during running or the output information after running from the communication unit 140A and transmitting text data or image data according to the output information during running or the output information after running to the display unit 170A. The processing unit 120A further performs a process of transmitting sound data according to the output information during running or the output information after running to the sound output unit 180A or a process of transmitting vibration data according to the output information during running to the vibration unit 190A.

The processing unit 120A further performs, for example, a process of generating time image data according to time information received from the clocking unit 160A and transmitting the time image data to the display unit 170A.

For example, the storage unit 130A is configured by any of various IC memories such as a ROM storing data or programs used for the processing unit 120A to perform various processes or a RAM serving as a work area of the processing unit 120A.

The communication unit 140A performs data communication with the communication unit 40A of the exercise ability calculation apparatus 2A. Specifically, the communication unit 140A performs a process of receiving a command (for example, a command of the measurement start/the measurement stop or a command to start/end the running analysis process) according to operation data from the processing unit 120A and transmitting the command to the exercise ability calculation apparatus 2A. The communication unit 140A further performs a process of receiving the output information during running, the output information after running, or the exercise ability evaluation information transmitted from the exercise ability calculation apparatus 2A and transmitting the received information to the processing unit 120A, or transmits the measurement data of the pulse rate output from the pulsimeter 70 to the exercise ability calculation apparatus 2A.

The operation unit 150A performs a process of acquiring operation data (operation data of the measurement start/the measurement stop, selection of display content, or the like) from the user and transmitting the operation data to the processing unit 120A. The operation unit 150A may be, for example, a touch panel type display, a button, a key, or a microphone.

A process of acquiring basic data such as information regarding the weight or the like of the user used for the exercise ability calculation process according to the embodiment may be acquired by the operation unit 150A and transmitting the basic data to the processing unit 120A may be performed. The basis data obtained by the operation unit 150A and transmitted to the processing unit 120A is transmitted to the exercise ability calculation apparatus 2A via the communication unit 140A.

The clocking unit 160A performs a process of generating time information of year, month, day, hour, minute, second, or the like. For example, the clocking unit 160A is realized by a real time clock (RTC) IC or the like.

The display unit 170A displays image data or text data transmitted from the processing unit 120A as text, a graph, a table, animation, or another image. For example, the display unit 170A may be realized by a display such as a liquid crystal display (LCD), an organic electroluminescence (EL) display, an electrophoretic display (EPD) and may be a touch panel type display. The functions of the operation unit 150A and the display unit 170A may be realized by one touch panel type display.

The sound output unit 180A outputs the sound data transmitted from the processing unit 120A as sound such as voice or buzzer sound. For example, the sound output unit 180A is realized by a speaker, a buzzer, or the like.

The vibration unit 190A vibrates according to the vibration data transmitted from the processing unit 120A. The vibration is delivered to the display apparatus 3A so that the user wearing the display apparatus 3A can feel the vibration. For example, the vibration unit 190A is realized by a vibration motor or the like.

### Exercise Ability Calculation Method (Procedure of Process)

Next, an exercise ability calculation method for the user will be described with reference to the drawings. In the embodiment, an exercise ability calculation method when the user runs will be described. FIG. 8 is a flowchart illustrating an example of a procedure of the exercise ability calculation process (which is an example of an exercise ability calculation method) performed by the processing unit 20A. The processing unit 20A performs the exercise ability calculation process in the procedure of the flowchart of FIG. 8 by executing the exercise ability calculation program 370 stored in the storage unit 30A.

In FIG. 8, the processing unit 20A waits until the command of the measurement start is received (N in step S110). When the command of the measurement start is received (Y in step S110), the processing unit 20A first calculates an initial posture, an initial position, and an initial bias using the sensing data and the GPS data measured by the inertial measurement unit 10A on the assumption that the user stops, generates data for requesting the user to start running with his or her utmost effort, and transmits the data to the display apparatus 3A (step S120). Of the text data, the image data, the sound data, and the vibration data above described, the data for requesting the user to start the running is preferably the sound data or the vibration data by which the user can recognize that the user is requested to start the running even when the user does not continuously watch the display unit 170A. However, the text data or the image data may be displayed by the display apparatus 3A. The user starts the running for the exercise ability calculation process when the user recognizes the data for requesting the user to start the running through sound, vibration, or the like from the display apparatus 3A.

The processing unit 20A acquires the sensing data from the inertial measurement unit 10A during the running of the user started in step S120. The sensing data includes acceleration, an angular velocity, a velocity, a position, a posture angle, a distance, a stride, and a running pitch calculated by performing calculation using the outputs of the acceleration sensor 12A and the angular velocity sensor 14A, the GPS data obtained from the detection result of the GPS unit 50A, and geomagnetism data obtained from the detection result of the geomagnetic sensor 60A. The sensing data is acquired by the measurement data acquisition unit 21A and is chronologically added to the sensing data table 310A of the storage unit 30A (step S130).

The processing unit 20A acquires information regarding the weight of the user (step S140). As described above, the information regarding the weight of the user is input in advance by operating the operation unit 150A of the display apparatus 3A, is transmitted to the exercise ability calculation apparatus 2A, and is stored in the storage unit 30A. A timing at which the information regarding the weight of the user is acquired may not be a timing after the user starts running in step S120.

The measurement data acquisition unit 21A of the processing unit 20A acquires information regarding the pulse rate measured by the pulsimeter 70 of the display apparatus 3A (step S150).

Next, the processing unit 20A causes the exercise ability data generation unit 260 to generate exercise ability data of the user. When the exercise ability data generation unit 260 generates the exercise ability data, an exercise output indicating an exercise amount within a predetermined time is first calculated from the sensing data including the acceleration and the velocity acquired in step S130 and the information regarding the weight of the user (step S160).

Next, the exercise ability data generation unit 260 calculates a cardiac output based on the pulse rate of the user acquired in step S150 and the exercise output calculated in step S160 (step S170). A relation among a cardiac output SV, a pulse rate HR, and an exercise output can be expressed in an exercise output=a (SV×HR). Here, a is a coefficient which is based on the information regarding the weight or the like described above. Thus, the cardiac output can be estimated based on a value obtained by dividing the exercise output SV by the pulse rate HR.

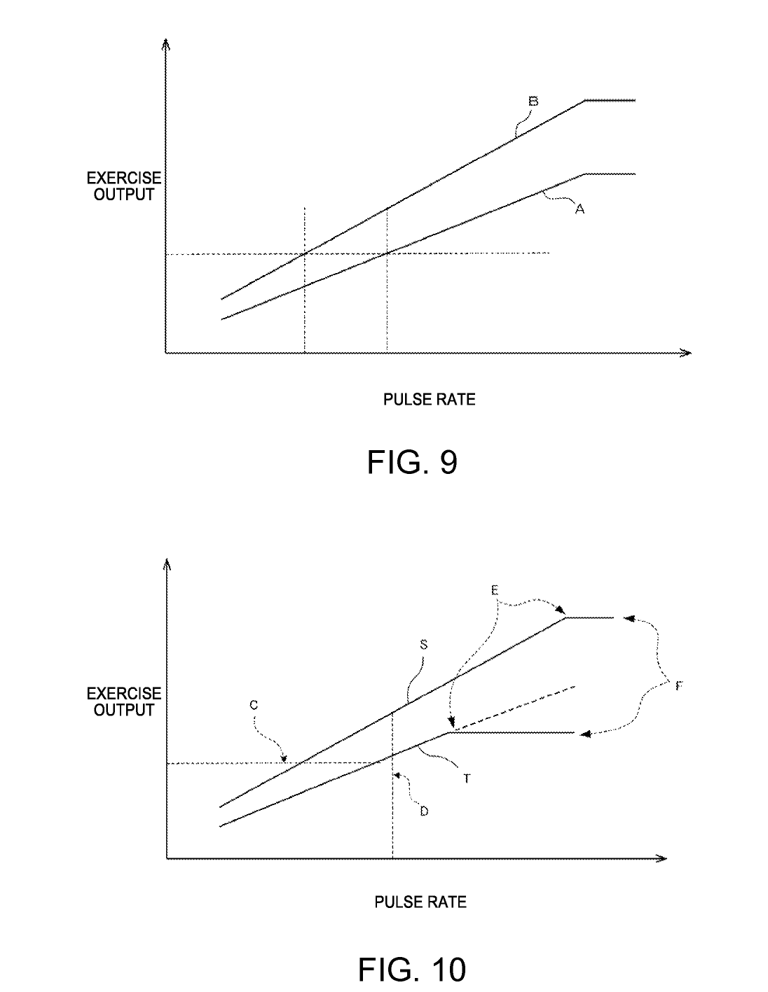

Next, the processing unit 20A evaluates an exercise ability of the user based on the exercise ability data generated in step S170 (step S180). FIG. 9 is a diagram for describing an example of evaluation of an exercise ability of a user in an exercise ability calculation process.

In FIG. 9, the vertical axis represents the exercise output and the horizontal axis represents the pulse rate. In the graph indicating the relation between the indexes on the two axes, data B indicating the exercise ability of the user at this time and data A indicating the exercise ability of the user at a previous time or a past exercise ability of the user are plotted.

When the values of the pulse rates at the same exercise output of the exercise ability data B of the user generated at this time and the exercise ability data A of the user at the previous time or the previous exercise ability data A are compared in FIG. 9, a result in which the pulse rate of the exercise ability data B at this time is smaller than the pulse rate of the exercise ability data A at the previous time or the previous exercise ability data A is obtained. This demonstrates that the pulse rate when the user does an exercise of the exercise output of the same intensity is lowered, and thus it can be evaluated that the exercise ability is improved. The same evaluation can be performed even when exercise outputs at the same pulse rate are compared.

Referring back to FIG. 8, next, the processing unit 20A determines whether the user is notified of the result of the exercise ability evaluation of step S180 (step S190). When it is determined that it is not necessary to notify the user of the result of the exercise ability evaluation (N in step S190), the exercise ability calculation process ends. Unlike this determination, when the processing unit 20A determines that the user is notified of the result of the exercise ability evaluation, for example, when the exercise ability is improved over a certain range or the exercise ability is degraded oppositely in the result of the exercise ability evaluation illustrated in FIG. 9 (Y in step S190), the processing unit 20A generates notification data (advice data) from a kind of desired data of which the user is notified, according to the result of the exercise ability evaluation (step S200) and transmits the notification data to the display apparatus 3A via the communication unit 40A (step S210). As the notification data, for example, various kinds of notification are considered, such as messages used to notify the user of exercise ability information indicating an improvement in the exercise ability at the time of training at a previous time or necessity of training for improving a muscular strength, endurance, or the like.

The user may be notified of the result of the exercise ability evaluation as the notification data in real time.

After the user is notified of the notification data, a series of exercise ability calculation processes ends.

In the exercise ability calculation method according to the fifth embodiment described above, presence or absence or the magnitude (degree) of the change in the exercise ability of the user has been comprehended by comparing the data indicating the relation between the pulse rate and the exercise output calculated at this time to the data indicating the relation between the pulse rate and the past exercise output, but the invention is not limited thereto. As another exercise ability calculation method, for example, a cardiac output of the user obtained in the exercise ability calculation process of the foregoing fifth embodiment may be compared to a past cardiac output of the user or a reference value of a cardiac output of the same sex and the same age, and the exercise ability of the user may be evaluated based on the comparison result. Further, the maximum oxygen intake amount can also be estimated from the cardiac output and the pulse rate obtained in the exercise ability calculation method of the fifth embodiment.

As described above, in the exercise ability calculation apparatus 2A, the exercise ability calculation system 1A, and the exercise ability evaluation method using the apparatus and the system according to the fifth embodiment, it is possible to obtain the following advantages.

(1) In the exercise ability calculation system 1A and the exercise ability calculation process (the exercise ability calculation method) using the system according to the fifth embodiment, the relative evaluation result of the exercise ability of the user can be presented to the user. Thus, it is possible to expect advantages of denoting the change or the degree of improvement or degradation of the exercise ability of the user and causing the user to recognize the change or the degree, and thus giving a hint to the user who decides a subsequent training method or achieving an improvement in a motivation for the training of the user.

(2) The exercise ability calculation system 1A according to the fifth embodiment includes the exercise ability calculation apparatus 2A including the inertial measurement unit 10A that includes the inertial sensor worn by the subject, the measurement data acquisition unit 21A that serves as the reception unit acquiring the information regarding the acceleration and the velocity from the output of the inertial sensor, the information regarding the weight of the subject, and the like, and the exercise ability data generation unit 260 that serves as the calculation unit calculating the exercise output of the subject within the predetermined time from the information regarding the acceleration, the velocity, and the weight.

Thus, the exercise output indicating an exercise amount of the subject within the predetermined time can be calculated with high accuracy from the information regarding the acceleration and the velocity acquired by the inertial sensor worn by the user and the information regarding the weight of the subject, and thus it is possible to provide the exercise ability calculation system 1A capable of evaluating the exercise ability of the subject based on the calculated exercise output.

(3) The exercise ability calculation system 1A according to the fifth embodiment has been configured to include the pulsimeter that measure a pulse rate of the subject and to have a function of calculating a cardiac output based on the pulse rate of the subject measured by the pulsimeter and the above-described exercise output. The pulsimeter has been configured to be mounted on the wrist type display apparatus 3A and measure a pulse rate of the wrist portion of the subject.

In this configuration, it is possible to calculate the cardiac output based on the pulse rate and the exercise ability of the subject and relatively easily calculate the exercise ability including the heart competence of the subject using the cardiac output. By configuring the pulsimeter as a type of pulsimeter mounted on the wrist type display apparatus 3A, for example, it is possible to continuously obtain measurement data of pulse values normally while reducing wearing inconvenience of the subject in comparison to a heart rate sensor or the like wound and worn around a chest.

(4) The exercise ability calculation method according to the embodiment has been configured to include the steps of acquiring the information regarding the acceleration and the velocity from the output of the inertial measurement unit 10A worn by the user, acquiring the information regarding the weight of the user, and calculating the exercise output indicating the exercise amount within the predetermined time from the acquired information.

Thus, it is possible to calculate the exercise output indicating the exercise amount of the user within the predetermined time relatively simply and with high accuracy, and thus to evaluate the exercise ability of the user based on the calculated exercise output.

(5) In the embodiment, the exercise ability calculation method has been configured to include the steps of acquiring the information regarding the pulse rate by the pulsimeter worn by the user and calculating the cardiac output based on the pulse rate and the exercise output.

Thus, it is possible to calculate the cardiac output based on the pulse rate and the exercise ability while acquiring the pulse rate of the user in real time and with high accuracy, and to calculate the exercise ability including the heart competence of the subject using the cardiac output.

(6) In the embodiment, the cardiac output has been configured to be estimated based on the value obtained by dividing the exercise output by the pulse rate.

Thus, it is possible to relatively easily estimate the cardiac output, for which a special equipment or mechanism is normally necessary, from the exercise output calculated based on information or the like acquired by the inertial sensor and the pulse rate measured by the pulsimeter 70, and thus to calculate the exercise ability of the user based on the obtained cardiac output.

The invention is not limited to the above-described fifth embodiment, but modifications, improvements, or the like of the above-described embodiment can be made. Other embodiments of the foregoing embodiment will be described below.

### Other Embodiments

The invention is not limited to the above-described fifth embodiment, but modifications, improvements, or the like of the above-described embodiment can be made. Other embodiments of the foregoing embodiment will be described below. The same reference numerals are given to the same constituent elements as the fifth embodiment, and the repeated description will be omitted.

### Sixth Embodiment

FIG. 10 is a diagram for describing an example of evaluation of an exercise ability of a user in an exercise ability calculation process of a sixth embodiment.

In FIG. 10, the vertical axis represents the exercise output and the horizontal axis represents the pulse rate. In the graph indicating the relation between the indexes on the two axes, first data S indicating the exercise ability of the user at this time and second data T indicating the exercise ability of the user at a previous time or a past exercise ability of the user are plotted.

In FIG. 10, in a broad sense, there are four points for comparison evaluation between the first data S which is exercise ability data of the user generated at this time and the second data T which is exercise ability data of the user at a previous time or a past exercise ability data of the user. Four comparison evaluation methods will be described below.

### C Portion

First, in a portion indicated by an arrow C of FIG. 10, pulse rates of the first data S and the second data T are compared at a predetermined exercise output. This is the same as the evaluation method performed in the foregoing fifth embodiment. That is, when the values of the pulse rates of the first data S and the second data T at the predetermined same exercise output are compared, the pulse rate of the first data S is smaller than the pulse rate of the second data T. The fact that the pulse rate obtained at the time of the exercise of the exercise output of the same intensity is smaller can be said that the exercise ability in the first data S is higher than in the second data T. Thus, the exercise ability can be determined to be higher as the pulse rate at the same exercise output is lower, and thus the exercise ability of the subject can be comprehended relatively.

### D Portion

Next, in a portion indicated by an arrow D of FIG. 10, the exercise outputs of the first data S and the second data T at a predetermined pulse rate are compared in contrast to the above-described arrow C. When the values of the exercise outputs of the first data S and the second data T at the predetermined same pulse rate are compared, the exercise output of the first data S is larger than the exercise output of the second data T, as in the C portion. Accordingly, the exercise ability in the first data S in which a stronger exercise output is obtained in a state of the smaller pulse rate can be determined to be higher than in the second data T. Thus, it is possible to relatively comprehend the exercise ability of the subject.

### E Portion

Next, in portions (two portions) indicated by an arrow E of FIG. 10, pulse rates at which the exercise outputs of the first data S and the second data T are saturated are compared. The fact that the exercise output is saturated refers to a state in which the exercise output does not increase despite an increase in the pulse rate when the pulse rate increasing linearly in substantial proportion to the increase in the exercise output reaches a certain value. When the exercise output is saturated, the exercise output can decrease despite the increase in the pulse rate. In FIG. 10, the exercise output of the second data T is saturated at the smaller pulse rate than the first data S. Thus, the exercise ability of the second data T can be determined to be lower than that of the first data S in that a point at which the exercise output is not set to be appropriate for the pulse rate is low as the pulse rate at the time of the saturation of the exercise output is lower.

### F Portion

Next, portions indicated by an arrow F of FIG. 10 show the maximum pulse rates of the first data S and the second data T. In the embodiment, the maximum pulse rate is assumed to be the same as the maximum heart rate. In FIG. 10, the maximum pulse rate in the first data S is higher than in the second data T. In the embodiment, the exercise ability can be determined to be higher as the maximum pulse rate is higher.

### Seventh Embodiment

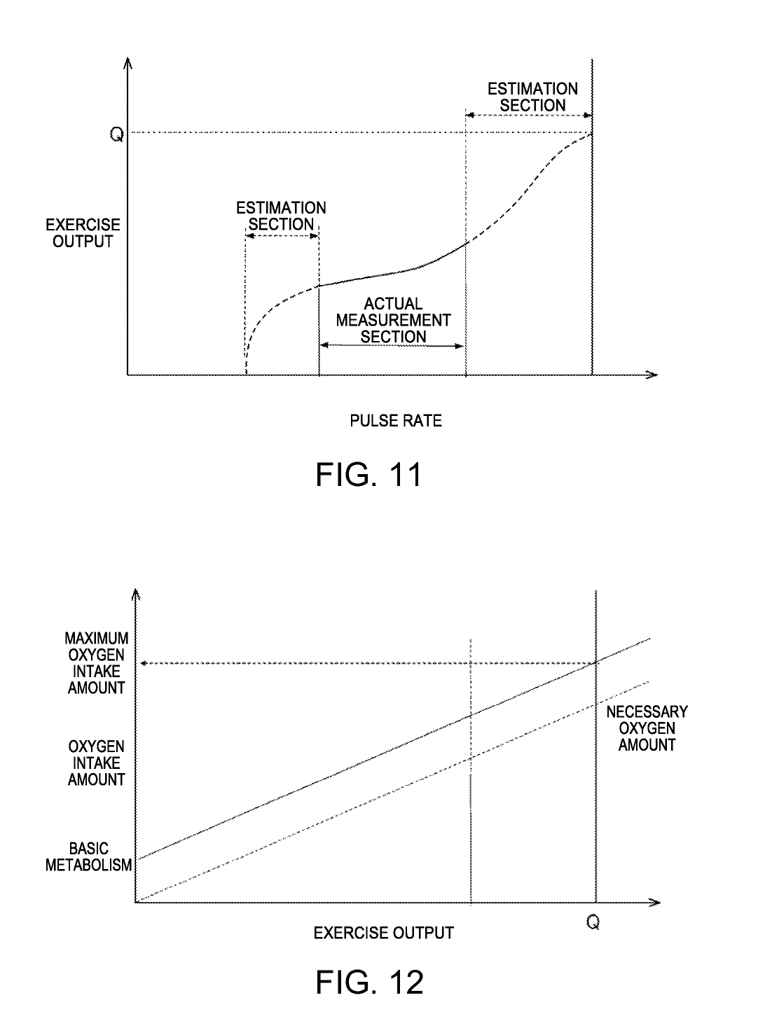

FIGS. 11 and 12 are diagrams for describing evaluation examples of the exercise ability of the user in the exercise ability calculation process according to a seventh embodiment. FIG. 11 is a diagram for describing a method of obtaining a relation between the pulse rate and the exercise output of an estimation section from a relation between a pulse rate and an exercise output of an actual measurement section. FIG. 12 is a diagram for describing a method of calculating an exercise ability of a subject from an estimation result illustrated in FIG. 11.

In data (graph) indicating the relation between the exercise output (represented by the vertical axis) and the pulse rate (represented by the horizontal axis) illustrated in FIG. 11, a graph line shown in the actual measurement section is calculated and plotted based on the sensing data or the pulse rate data actually measured in the exercise ability calculation method (steps S110 to S160 (S170) of FIG. 8) described in the foregoing fifth embodiment.

In contrast, graph lines of sections shown as estimation sections in the drawing are estimated from the inclination of the graph line in the actual measurement section.

Since the exercise output at a predetermined pulse rate can be estimated from estimation values (the graph lines) in the estimation sections, the exercise ability of the subject can be calculated.

Here, the maximum pulse rate can be estimated as a predetermined pulse rate. By assuming the maximum pulse rate as the predetermined pulse rate, a maximum exercise output estimation value Q can be estimated.

In FIG. 12, the vertical axis represents an oxygen intake amount and the horizontal axis represents an exercise output. An exercise ability calculation method of estimating a maximum oxygen intake amount from the maximum exercise output estimated in FIG. 11 is shown. Specifically, an oxygen intake amount corresponding to the maximum exercise output estimation value Q calculated in FIG. 11 is estimated as the maximum oxygen intake amount. Thus, the maximum oxygen intake amount which is an effective index of the exercise ability evaluation can be calculated.

The embodiment of the invention devised by the present inventors have been described in detail above. However, the invention is not limited to the foregoing embodiments and may be modified in various ways within the scope of the invention without departing from the gist of the invention.

The entire disclosure of Japanese Patent Application No. 2014-155939, filed Jul. 31, 2014 and No. 2014-156772, filed Jul. 31, 2014 are expressly incorporated by reference herein.

What is claimed is:

1. An exercise ability evaluation method comprising:
acquiring measurement data of whole body endurance of a user;
acquiring reference data of the whole body endurance;
comparing the measurement data to the reference data; and
evaluating a muscle ability of the user based on a comparison result obtained by comparing the measurement data and the reference data and generating exercise ability evaluation information.

2. The exercise ability evaluation method according to claim 1, wherein an index of the whole body endurance includes at least one of a cardiorespiratory ability, a lactic acid value, a degree of fatigue and an oxygen intake amount of the user.

3. The exercise ability evaluation method according to claim 1, wherein the index is a running time per predetermined distance of the user.

4. The exercise ability evaluation method according to claim 2,
wherein the index is the oxygen intake amount, and
wherein the oxygen intake amount is measured through a running test within a predetermined time or a running test of reciprocation of a predetermined distance.

5. The exercise ability evaluation method according to claim 2,
wherein the index is the oxygen intake amount, and
wherein the oxygen intake amount is calculated based on pulse data and body movement data of the user.

6. The exercise ability evaluation method according to claim 4, wherein the oxygen intake amount serving as the reference data is acquired based on a heart rate at a time of rest and a maximum heart rate (HRₘₐₓ).

7. The exercise ability evaluation method according to claim 1, wherein the muscle ability of the user is evaluated based on a difference between the measurement data and the reference data and the exercise ability evaluation information is generated.

8. An exercise ability evaluation apparatus comprising:
a storage unit that stores reference data of whole body endurance; and
an exercise ability evaluation information generation unit that generates exercise ability evaluation information by evaluating a muscle ability of a user based on a comparison result obtained by comparing measurement data of whole body endurance of the user and the reference data.

9. An exercise ability calculation method comprising:
acquiring information regarding acceleration and velocity from an output of an inertial sensor worn by a subject;
acquiring information regarding a weight of the subject; and
calculating an exercise output indicating an exercise amount within a predetermined time from the information regarding the acceleration, the velocity, and the weight.

10. The exercise ability calculation method according to claim 9, further comprising:
acquiring information regarding a pulse rate of the subject; and
calculating a cardiac output based on the pulse rate and the exercise output.

11. The exercise ability calculation method according to claim 10, wherein information regarding the pulse rate is a pulse rate measured by a pulsimeter worn by the subject.

12. The exercise ability calculation method according to claim 10, wherein the cardiac output is estimated and calculated based on a value obtained by dividing the exercise output by the pulse rate.

13. The exercise ability calculation method according to claim 10, wherein an exercise ability is calculated based on a comparison result obtained by comparing the calculated cardiac output to a reference cardiac output to be compared to the cardiac output.

14. The exercise ability calculation method according to claim 10, wherein an exercise ability is calculated based on a result obtained by comparing first data indicating a relation between the pulse rate and the exercise output to second data indicating a relation between a different pulse rate from the first data and the exercise output.

15. The exercise ability calculation method according to claim 14, wherein the exercise outputs of the first data and the second data are compared at a predetermined pulse rate.

16. The exercise ability calculation method according to claim 14, wherein pulse rates of the first data and the second data are compared for a predetermined exercise output.

17. The exercise ability calculation method according to claim 14, wherein pulse rates at which the exercise outputs of the first data and the second data are saturated are compared.

18. The exercise ability calculation method according to claim 14, wherein maximum pulse rates of the first data and the second data are compared.

19. The exercise ability calculation method according to claim 9, wherein an inclination of a non-measurement section is estimated from an inclination of an actual measurement section of a graph indicating a relation between the pulse rate and the exercise output, and the exercise output at a predetermined pulse rate is estimated.

20. The exercise ability calculation method according to claim 19, wherein a maximum oxygen intake amount is estimated and calculated based on the estimated exercise output.

21. The exercise ability calculation method according to claim 19, wherein the predetermined pulse rate is a maximum pulse rate of the subject.

22. An exercise ability calculation apparatus comprising:
a reception unit that acquires information regarding acceleration and velocity from an output of an inertial sensor worn by a subject and information regarding a weight of the subject; and
a calculation unit that calculates an exercise output indicating an exercise amount within a predetermined time from the information regarding the acceleration, the velocity, and the weight.

23. The exercise ability calculation apparatus according to claim 22,
wherein the reception unit includes a function of acquiring information regarding a pulse rate of the subject, and
wherein the calculation unit includes a function of calculating a cardiac output based on the pulse rate and the exercise output.
